SEQUENCE LISTING (21 sequences)

SEQ ID NO: 1 - DNA, 519 bp, Homo sapiens
atggctttagagacgatctgccgaccctctgggagaaaatccagcaagatgcaagccttcagaatctgggatgttaaccagaagaccttctatctgaggaacaaccaactagttgccggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaa

SEQ ID NO: 2 - protein, 159 aa, Homo sapiens
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 3 - DNA, 1197 bp, Homo sapiens
atgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtga

SEQ ID NO: 4 - protein, 398 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 5 - DNA, 283 bp, Rattus norvegicus
gcaagccttcagaatctgggatactaaccagaagaccttctacctgaggaacaaccagctcattgctgggtacttacaaggaccaaataccaaactagaagaaaagatagacatggtgcctattgactttcggaatgtgttcttgggcatccacgggggcaagctgtgcctgtcttgtgtcaagtctggagatgacaccaagctccagctggaggaggttaacatcactgatctgaacaagaacaaagaagaagacaagcgctttaccttcatccgctccgag

SEQ ID NO: 6 - protein, 178 aa, Rattus norvegicus
MEICRGPYSHLISLLLILLFRSESAGHPAGKRPCKMQAFRIWDTNQKTFYLRNNQLIAGYLQGPNTKLEEKIDMVPIDFRNVFLGIHGGKLCLSCVKSGDDTKLQLEEVNITDLNKNKEEDKRFTFIRSETGPTTSFESLACPGWFLCTTLEADHPVSLTNTPKEPCTVTKFYFQEDQ

SEQ ID NO: 7 - DNA, 536 bp, Canis lupus familiaris
accgaatggaaacctgcaggtgtcctctcagctacctaatctctttcctccttttcctgtcccattcagagacagcctgccgtcccttggggaagagaccttgcaggatgcaagccttcagaatctgggatgttaaccagaagaccttctacctgaggaataaccaactagtcgctggatacttgcaaggatcaaatactaaattagaagagaagttagatgtggtgcccgtcgagcctcatgccgtgttcttggggatccatggggggaagctgtgcctggcctgtgtcaagtctggagatgagaccaggctccagctggaggccgttaacatcactgacctgagtaagaacaaggatcaagacaagcgctttaccttcatcctctcagacagtggccctaccaccagctttgagtctgctgcctgccctggctggttcctctgcacagcactggaggccgaccggcctgtcagcctcaccaacagaccagaagaggccatgatggtcactaagttctacttccagaaggaataa

SEQ ID NO: 8 - protein, 176 aa, Canis lupus familiaris
METCRCPLSYLISFLLFLSHSETACRPLGKRPCRMQAFRIWDVNQKTFYLRNNQLVAGYLQGSNTKLEEKLDVVPVEPHAVFLGIHGGKLCLACVKSGDETRLQLEAVNITDLSKNKDQDKRFTFILSDSGPTTSFESAACPGWFLCTALEADRPVSLTNRPEEAMMVTKFYFQKE

SEQ ID NO: 9 - DNA, 534 bp, Oryctolagus cuniculus
atgagaccctccaggagcacccgcaggcacctaatctccctcctcctcttcctgttccattcagagacagcctgccgcccttctgggaaaagaccttgcaggatgcaggccttcagaatctgggatgttaaccagaagaccttctacttgagaaacaaccaactagtcgctggttacttgcaaggcccaaatgccaaattagaagaaaggatagatgtggtgccccttgagcctcagctcctgttcctgggcatccagagggggaagttgtgcctgtcttgtgtgaagtctggggataagatgaagctccatttggaggccgttaacatcactgacctgggcaagaacaaggagcaggacaagcgcttcaccttcatccgctccaatagcggccctaccaccaccttcgagtctgcctcctgcccgggctggtttctctgcacggccctggaggctgaccagcccgtcagcctcaccaacacgcccgacgactccatcgtggtcaccaagttctacttccaggaggaccagtag

SEQ ID NO: 10 - protein, 177 aa, Oryctolagus cuniculus
MRPSRSTRRHLISLLLFLFHSETACRPSGKRPCRMQAFRIWDVNQKTFYLRNNQLVAGYLQGPNAKLEERIDVVPLEPQLLFLGIQRGKLCLSCVKSGDKMKLHLEAVNITDLGKNKEQDKRFTFIRSNSGPTTTFESASCPGWFLCTALEADQPVSLTNTPDDSIVVTKFYFQEDQ

SEQ ID NO: 11 - DNA, 534 bp, Equus caballus
atggaaatccgcaggcgttctgtcagacacctaatctctctcctccttttcttgctctactcagagacagcctgccaccctttggggaagagaccctgcaagatgcaagccttcagaatctgggatgttaaccagaagaccttctacatgaggaataaccaactagttgctggatacttgcaagaatcaaatactaaattacaagagaagatagatgtggtgcccattgagcctgatgctctattcctgggactccatgggaggaagctgtgcctggcctgtgtcaagtctggtgatgagattaggttccaattggaggcagttaacatcactgacctgagcaagaacaaggaggagaacaagcgcttcaccttcatccgctcaaacagtggccccaccaccagcttcgagtctgccgcctgccctggctggttcctctgcacggcgcaggaggcagaccggcccgtcagcctcaccaacaagcccaaagagtccttcatggtcaccaagttctacctccaggaggaccagtag

SEQ ID NO: 12 - protein, 177 aa, Equus caballus
MEIRRRSVRHLISLLLFLLYSETACHPLGKRPCKMQAFRIWDVNQKTFYMRNNQLVAGYLQESNTKLQEKIDVVPIEPDALFLGLHGRKLCLACVKSGDEIRFQLEAVNITDLSKNKEENKRFTFIRSNSGPTTSFESAACPGWFLCTAQEADRPVSLTNKPKESFMVTKFYLQEDQ

SEQ ID NO: 13 - DNA, 534 bp, Sus scrofa
atggaagttagcaggtatctgtgcagttacctaatctctttcctccttttcctgttccactcagagactgcctgccaccccttgggaaagagaccttgcaggatgcaagccttcagaatctgggatgtcaaccagaagactttctacctgaggaataaccaattagttgctggatacttgcaaggaccaaatactaaactggaagagaagatagatgtggtgcctgttgagcctcattttgtgttcctggggatccatggagggaagctgtgcctgtcctgtgtcaagtctggtgatgagatgaagctccagttggacgcagttaacatcacagacctgagaaagaacagcgagcaggacaagcgcttcaccttcatccgctccgacagtggccccaccaccagctttgagtcagccgcctgtcctggctggttcctctgcactgcactagaggcagaccagcctgttggcctcaccaacacgcccaaagcagccgtcaaggtcaccaagttctacttccagcaggaccagtaa

SEQ ID NO: 14 - protein, 177 aa, Sus scrofa
MEVSRYLCSYLISFLLFLFHSETACHPLGKRPCRMQAFRIWDVNQKTFYLRNNQLVAGYLQGPNTKLEEKIDVVPVEPHFVFLGIHGGKLCLSCVKSGDEMKLQLDAVNITDLRKNSEQDKRFTFIRSDSGPTTSFESAACPGWFLCTALEADQPVGLTNTPKAAVKVTKFYFQQDQ

SEQ ID NO: 15 - DNA, 480 bp, Mus musculus
atggcttcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagttagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaatag

SEQ ID NO: 16 - protein, 159 aa, Mus musculus
MASEAACRPSGKRPCKMQAFRIWDTNQKTFYLRNNQLIAGYLQGPNIKLEEKLDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 17 - DNA, 525 bp, Bos taurus
atggacatctacatccacggttacctaatctgtcttctcctcttcctgttccgttcagaaacagcctgccaccccttgggaaagagacgctgcgagatgcaagccttcaggatctgggatgtcaaccagaagatcttctacctgaggaataaccaattagttgctggatacttgcaaggaccaaatactaaattagaggagaagatagatgtggtacccatcgaaccccatactatgttcctggggatccatggggggaagctgtgcctggcctgcgtaaaatctggagatgagatcaagctcaagttagaggccgtgaacatcactgacttgaaccagaacagggagcaggacaagcgctttgccttcatccgcttcgacaacgggcccaccaccagctttgagtcagctgcctgccccggctggttcctctgcacatcactggaggccgaccagcccgtgggcctcaccaatatgcccacagaggccctcaaggtcaccaagttctacttccagcaggactag

SEQ ID NO: 18 - protein, 174 aa, Bos taurus
MDIYIHGYLICLLLFLFRSETACHPLGKRRCEMQAFRIWDVNQKIFYLRNNQLVAGYLQGPNTKLEEKIDVVPIEPHTMFLGIHGGKLCLACVKSGDEIKLKLEAVNITDLNQNREQDKRFAFIRFDNGPTTSFESAACPGWFLCTSLEADQPVGLTNMPTEALKVTKFYFQQD

SEQ ID NO: 19 - protein, 177 aa, Macaca fascicularis
MEICRGLGSHLICLLLFLFHSETVGRPSGRKPSKMQAFRIWDVNQKTFYLRNNQLVAGYLQGPNVNLEEKIDVVPIEPHALFLGIHGGKMCLSCVKSGDETRLQLEAVNITDLSKNRKQDKRFAFVRSDSGPTTSFESAACPGWFLCTAMEADQPVSLTNMPDKGVMVTKFYFQEDE

SEQ ID NO: 20 - protein, 159 aa, Macaca mulatta
MALETVGRPSGRKPSKMQAFRIWDVNQKTFYLRNNQLVAGYLQGPNVNLEEKIDVVPIEPHALFLGIHGGKMCLSCVKSGDETRLQLEAVNITDLSKNRKQDKRFAFVRSDSGPTTSFESAACPGWFLCTAMEADQPVSLTNMPDKGVMVTKFYFQEDE

SEQ ID NO: 21 - protein, 174 aa, Monodelphis domestica
MDLSVCPRSYLITLFLFLFHSETESFPFVKMSSKRQAYRIWDIYQKTFYLRNNQLVAGYLQGSNTNLEEKIDLVSIEPDSVLLGIHDGRLGLACVKTGGTPKLQLEAVNITDLDKNQQESKRFTFIKSENDSICRFESAACPGWFLCTQTESDRPVGLSNTQANTITRFYFQRE

(19) United States
(12) Patent Application Publication
(10) Pub. No.: US 2008/0187576 A1
(43) Pub. Date: Aug. 7, 2008

(54) METHODS FOR TREATING ARTICULAR DISEASE OR DYSFUNCTION USING SELF-COMPLIMENTARY ADENO-ASSOCIATED VIRAL VECTORS

(75) Inventors: Steven Craig Ghivizzani, Gainesville, FL (US); Christopher Howard Evans, Cohasset, MA (US); Elvire Gouze, Gainesville, FL (US); Jean-Noel Gouze, Gainesville, FL (US)

Correspondence Address: HAYNES AND BOONE, LLP, 901 Main Street, Suite 3100, Dallas, TX, 75202, US

(73) Assignees: UNIVERSITY OF FLORIDA RESEARCH FOUNDATION, INC., Gainesville, FL (US); THE BRIGHAM AND WOMEN'S HOSPITAL, INC., Boston, MA (US)

(21) Appl. No.: 11/764,771

(22) Filed: Jun. 18, 2007

Related U.S. Application Data
(60) Provisional application No. 60/814,468, filed on Jun. 16, 2006.

(51) Int. Cl. A61K 48/00; A61K 9/127; A61K 31/7088; A61P 19/02; A61K 9/14

(52) U.S. Cl. 424/450; 424/490; 424/489; 424/93.2; 514/44

(57) ABSTRACT

Disclosed are methods and compositions for treating articular diseases such as osteoarthritis and rheumatoid arthritis, or articular dysfunction in mammals. Also disclosed are self-complimentary AAV vectors useful in gene therapy and delivery of therapeutic constructs to mammalian cells, and in particular, articular joints, cartilage, ligaments, tendons, and surrounding tissues.

The present application claims priority to U.S. Patent Application Ser. No. 60/814,468, filed Jun. 16, 2006, the entire contents of which is specifically incorporated herein by reference.

The United States government has certain rights in the present invention pursuant to grant number 1R01-AR48566 from the National Institutes of Health.

### 1. BACKGROUND OF THE INVENTION

1.1. Field of the Invention

The present invention relates generally to the fields of medicine and viral vector based gene therapy, and in particular, to the development of self-complementary adeno-associated viral (scAAV) vectors and compositions comprising them, for use in methods of treating articular diseases and dysfunctions including, for example, osteoarthritis and rheumatoid arthritis, in affected mammals.

1.2. Description of Related Art

### 1.2.1 Anti-Arthritic Proteins for Treating Articular Disease

Arthritic diseases are common crippling conditions for which there are no cures. Recent research into the biology of rheumatoid arthritis (RA) and osteoarthritis (OA), the two most common forms of arthritis, has identified a number of potentially anti-arthritic proteins with promising anti-inflammatory, immunomodulatory or chondroprotective properties. A number of these proteins have been evaluated in animal models of arthritis and in clinical trials with encouraging results.³

Proteinaceous agents, in general, however, have limited half-lives in vivo, and thus require repeated administration to maintain therapeutic efficacy. Recombinant proteins are also expensive to manufacture and therefore costly to consumers. Prime examples are the commercially-available TNF inhibitors, etanercept and infliximab (Enbrel™ and Remicade™, respectively), and interleukin-1 (IL-1) receptor antagonist (IL-1Ra), anakinra (Kineret™, Amgen), which have all proven effective in treating some patients with RA, but, unfortunately require frequent injection(s) and/or infusion(s) (e.g., daily in the case of Kineret™), and may cost the patient more than $10,000 to $12,000 annually. Moreover, systemic route of delivery of such peptide therapeutics may engender untoward side effects.

Intra-articular delivery of these therapeutic molecules is also exacerbated by the fact that articular cartilage, the main site of pathology in OA and a primary drug target, is avascular and alymphatic. Substances enter cartilage via diffusion from the synovial fluid, itself a dialysate of the peripheral blood. Access to chondrocytes is further constrained by the highly anionic, dense, extracellular matrix that surrounds these cells. Even in advanced arthritis where substantial degradation of this matrix has occurred, molecules do not have free access to chondrocytes. Other intra-articular structures that are damaged in OA, such as ligaments and menisci, are also poorly vascularized.

These physiological and anatomical constraints bear important consequences for drug delivery to joints. Although small molecules administered parenterally diffuse into the synovial fluid and thence to intraarticular tissues, they also travel to other organs, so that treatment of an articular problem comes with the risk of unwanted side-effects. Delivery of larger molecules, such as proteins, is even more problematic, as, depending on their size, they have difficulty entering the joint. Direct, intraarticular injection ensures delivery of large molecules to joints, but these molecules are rapidly cleared via the lymphatics.

### 1.2.2 Gene Therapy for Treating Articular Disease

In its most fundamental form, arthritis gene therapy involves the transfer to the body of complementary DNA (cDNA) encoding one or more antiarthritis gene products that are otherwise difficult to administer in a sustained or targeted manner (excerpted from the review by Evans et al⁶³). These include proteins that act as cytokine antagonists, immunomodulators, growth factors, transcription regulators, enzyme inhibitors, antioxidants, and antinociceptive agents.

In a related strategy that does not necessarily require long-term transgene expression, the cDNA may encode a product that induces apoptosis or in some other manner eliminates cells that contribute to disease. Alternatively, the cDNA may encode a therapeutic species of RNA, such as a ribozyme, antisense RNA, or a small interfering RNA molecule. Extensions of these concepts include the delivery of the RNA itself or non-coding nucleotide sequences that act, for example, as decoys, and the use of DNA to vaccinate against arthritis.

Transfer of nucleic acids into target cells can be accomplished by nonviral (transfection) or viral (transduction) means. The leading viral vectors used in clinical trials in humans are oncoretrovirus (usually referred to just as retrovirus), lentivirus (also a member of the retrovirus family), adenovirus, adeno-associated virus (AAV), and herpes simplex virus (HSV).

Intraarticular gene delivery has the advantage of localizing the material at the major sites of disease activity, the joints, and minimizing exposure of non-target tissues, thereby reducing unwanted side effects. The safety of this approach is reinforced by results from the first clinical trial and by data from tracking studies confirming that, under most conditions, the transgene remains largely within the joint.

Because less gene product (and hence, less vector) is needed to treat an individual joint compared to that required to treat the whole body, gene therapy also represents the most economical approach. For example, Makarov et al.⁶² has calculated that the IL-1Ra synthesized locally as a result of intra-articular gene transfer is 10,000 times more potent than recombinant IL-1Ra delivered systemically.

1.3. Deficiencies in the Prior Art

The drawbacks of direct local delivery of adenoviral vectors, however, are also not without consequence. To date, most articular-directed gene therapy vectors have been adenoviral based. Unfortunately, these vectors are often responsible for various side-effects, and are therefore unsuited for administration to many patients.

Although a variety of gene delivery vehicles have been developed over the years to overcome the aforementioned limitations, none has been particularly suited for gene delivery to the joints and the treatment of articular diseases. Non-viral systems are currently inefficient and provide only transient transgene expression, often accompanied by inflammation. Adenoviral vectors can often enable high levels of the gene transfer in certain tissue types, but are associated with inflammatory reactions that lead to transient transgenic expression. Lentiviral vectors are highly efficient, but because they are integrating have the potential to cause insertional mutagenesis. Herpes simplex virus, which can enable efficient gene transfer in many systems, are often cytotoxic and express native viral proteins which lead to immune clearance of modified cells.

Even prior studies involving the use of single-stranded forms of adeno-associated viruses have been very disappointing, due largely to the relatively inefficient expression of the therapeutic gene encoded by single-stranded AAV vector constructs.

In fact, among the ssAAV vectors tested to date, none has been capable of achieving sustained, biologically-relevant levels of the encoded therapeutic agent intra-articularly, even when directly injected into the joint space. The poor efficiency of synthesis of the second DNA strand of the typical wild-type AAV vectors has largely been implicated as the primary reason for the limited success seen with traditional AAV-based therapies.

Moreover, the low-levels of resulting gene expression following injection of traditional single-stranded viral vectors into the joint space of affected mammals, coupled with the slow onset of transgenic expression (typically one to two weeks' minimum), and the resulting sub-therapeutic levels of expression have significantly limited the successful exploitation of conventional viral vector-based gene therapies for the treatment of articular diseases and dysfunctions.

There is a need, therefore, for the creation of vectors and compositions that can be used in appropriate therapeutic regimens to achieve safe, sustained, local, intraarticular, delivery of therapeutic agents to mammalian joints.

### 2. SUMMARY OF THE INVENTION

The present invention overcomes these and other limitations inherent in the prior art by providing new scAAV-based genetic constructs that encode one or more mammalian therapeutic agents for the prevention, treatment, and/or amelioration of one or more symptoms of a disorder, disease, or dysfunction, that results from one or more altered or abnormal activities or metabolic conditions in a host cell, and in particular, from overexpression of IL-1 receptor polypeptide in cells and/or tissues of joints and the periarticular space of a mammal. In particular embodiments, the invention provides means for directly providing to such tissues a viral vector encoding a biologically-active IL-1Ra polypeptide whose expression persists well after initial administration of the vector composition to the affected joints/tissues/periarticular space.

In an effort to develop a more effective and enduring drug-delivery strategy, and to overcome the limitations associated with adenoviral-based therapeutic regimens proposed by others, the present inventors have exploited for the first time self-complimentary, double-stranded, scAAV vectors useful in the treatment of arthritis and other joint pathologies.¹ By delivering therapeutic constructs with anti-arthritic properties to cells in the synovial lining of joints and enabling persistent production and secretion of the encoded molecules, it is possible to maintain therapeutic levels in the joint for extended periods. By stably inserting exogenous therapeutic polynucleotides into these tissues, it is now possible to attain long-term relief, or even to reverse the pathologies of arthritic disease.

By developing an effective intra-articular method of scAAV-mediated gene delivery, a local approach is provided wherein exogenous genes are delivered to cells within the synovium of specific joints, where the protein products are able to be synthesized within the joint capsule¹ permitting the highest concentration of the protein at the actual site of the injury, disease, or dysfunction. This localized administration methodology significantly reduces the problems associated with systemic administration of therapeutics—namely, the risk of exposure of unafflicted tissues and organs. Also, the small fluid volume of the joint space relative to the total human blood volume requires significantly less polypeptide to be synthesized in order to achieve a localized therapeutic concentration of the composition. Moreover, the methods developed herein for the transfer of nucleic acid segments encoding mammalian Il-1Ra polypeptide species to selected, specific joints can now be applied to the treatment of both RA and OA in particular, in which typically only a limited number of joints are affected.

The present invention provides compositions comprising recombinant self-complementary (i.e., double-stranded) adeno-associated viral (AAV) vectors (scAAV), virions, viral particles, and pharmaceutical formulations thereof, useful in methods for delivering genetic material encoding one or more beneficial or therapeutic product(s) to mammalian cells and tissues. In particular, the compositions and methods of the invention provide a significant advancement in the art through their use in the treatment, prevention, and/or amelioration of symptoms of one or more mammalian articular diseases.

In one embodiment, the invention concerns scAAV-based genetic constructs that encode one or more mammalian therapeutic polypeptides for the prevention, treatment, and/or amelioration of various disorders resulting from a deficiency in one or more of such polypeptides. In particular, the invention provides scAAV-based genetic constructs encoding one or more mammalian therapeutic proteins, polypeptides, or peptides, as well as variants, and/or active fragments thereof, for use in the treatment, prophylaxis, and/or amelioration of one or more symptoms of a variety of conditions and mammalian diseases, dysfunctions, injuries, and/or disorders, including for example, articular damage, dysfunction, or disease, as well as diseases, dysfunctions, or disorders of the skeletal system, including for example, the joints or surrounding joint tissues, cartilage, tendons, ligaments, vertebral discs, disc space, a vertebral annulus, synovium, subsynovium, joint capsule, and/or periarticular muscle(s).

In another embodiment, the invention concerns scAAV vectors that comprise a nucleic acid segment that encodes one or more therapeutic agents that alter, inhibit, reduce, or impair the activity of one or more endogenous biological processes in the cell. In particular embodiments, a desired therapeutic agent is one that inhibits or reduces the effects of one or more metabolic processes, injuries, or diseases that cause or result in an autoimmune disease, or that contribute to, or facilitate the development of a condition such as arthritis or other joint or articular defect. In certain embodiments, the defect may be caused by injury or trauma to the joint and/or surrounding tissues. In other embodiments, the defect may be caused the over-expression of an endogenous biological compound, while in yet other embodiments, the defect may be caused by the under-expression or even lack of one or more endogenous biological compounds.

When the use of such scAAV vectors is contemplated for introduction of one or more exogenous IL-1Ra proteins, polypeptides, or peptides into a particular cell or tissue transfected with the vector, one may employ the scAAV vectors disclosed herein by genetically modifying the vectors to further comprise at least a first exogenous polynucleotide operably positioned downstream and under the control of at least a first heterologous promoter that expresses the polynucleotide in a cell comprising the vector to produce the encoded therapeutic peptide, protein, and/or polypeptide.

Such constructs may employ one or more heterologous promoters to express the therapeutic Il-1Ra composition. Such promoters may be constitutive, inducible, or even cell- or tissue-specific. Exemplary promoters include, but are not limited to, a CMV promoter, a β-actin promoter, a hybrid CMV promoter, a hybrid M-actin promoter, an EF1 promoter, a U1a promoter, a U1b promoter, a Tet-inducible promoter, a VP16-LexA promoter, a joint-specific promoter and a human-specific promoter.

The scAAV vectors and expression systems of the invention may also further comprise a second nucleic acid segment that comprises, consists essentially of, or consists of, one or more enhancers, regulatory or transcriptional elements, to alter or effect transcription of the heterologous nucleic acid segment comprised within the scAAV vector that encodes the therapeutic IL-1Ra protein, peptide or polypeptide.

For example, the scAAV vectors of the present invention may further comprise a second nucleic acid segment that comprises, consists essentially of, or consists of, at least a first CMV enhancer, a synthetic enhancer, or a cell- or tissue-specific enhancer. The second nucleic acid segment may also further comprise, consist essentially of, or consist of one or more intron sequences, post-transcriptional regulatory elements, or such like. The vectors and expression systems of the invention may also optionally further comprise a third nucleic acid segment that comprises, consists essentially of, or consists of, one or more polylinker or multiple restriction sites/cloning region(s) to facilitate insertion of one or more selected genetic elements, polynucleotides, and the like into the scAAV vectors at a convenient restriction site.

In aspects of the invention, the exogenous polynucleotides that are comprised within one or more of the improved scAAV vectors disclosed herein are preferably of mammalian origin, with polynucleotides encoding polypeptides and peptides of human, primate, murine, porcine, bovine, ovine, feline, canine, equine, epine, caprine, or lupine origin being particularly preferred.

As described above, the exogenous polynucleotide will preferably encode one or more IL-1Ra proteins, polypeptides, or peptides, or a combination of one or more of these therapeutic agents. In fact, the exogenous polynucleotide delivered via the scAAV vector may also optionally encode a second therapeutic molecule-encoding nucleic acid segment. When such “combinational” gene therapies are desired, two or more different molecules may be produced from a single scAAV expression system, or alternatively, a selected host cell may be transfected with two or more unique scAAV expression systems, each of which may comprise one or more distinct polynucleotides that encode a therapeutic agent.

In other embodiment, the invention also concerns the disclosed scAAV vectors comprised within an infectious adeno-associated viral particle or virion, or pluralities thereof, which may also be further comprised within one or more pharmaceutical vehicles, formulated for administration to a mammal such as a human for therapeutic, and/or prophylactic gene therapy regimens. Such vectors, virus particles, virions, and pluralities thereof may also be provided in excipient formulations that are acceptable for veterinary administration to selected livestock, exotic or domesticated animals, pets, and the like.

The invention also concerns host cells that comprise at least one of the disclosed scAAV vectors or expression systems. Such host cells are particularly mammalian host cells, with human host cells being particularly highly preferred, and may be either isolated, in cell or tissue culture. In the case of genetically modified animal models, the transformed host cells may even be comprised within the body of a non-human animal itself.

In certain embodiments, the creation of recombinant non-human host cells, and/or isolated recombinant human host cells that comprise one or more of the disclosed scAAV vectors is also contemplated to be useful for a variety of diagnostic, and laboratory protocols, including, for example, means for the production of large-scale quantities of scAAV vectors as described herein. Such viral production methods are particularly desirable to obtain the often high-titer viral stocks required by many gene therapy protocols.

Compositions comprising one or more of the disclosed scAAV vectors, expression systems, infectious AAV particles, or host cells also form part of the present invention and particularly those compositions that further comprise at least a first pharmaceutically-acceptable excipient for use in therapy, and for use in the manufacture of medicaments for the treatment of one or more mammalian diseases. Such pharmaceutical compositions may optionally further comprise one or more diluents, buffers, liposomes, a lipid, a lipid complex; or the scAAV vectors may be comprised within a microsphere or a nanoparticle. Pharmaceutical formulations suitable for intramuscular, intravenous, or direct injection into an organ or tissue or a plurality of cells or tissues of a human or other mammal are particularly preferred, including for example, formulations that are suitable for direct injection into the joint, synovium, subsynovium, joint capsule, tendon, ligament, cartilage, periarticular muscle or an articular space of a mammal.

Other aspects of the invention concern self-complimentary, double-stranded adeno-associated virus, virions, and viral particles, as well as compositions, and host cells that comprise, consist essentially of, or consist of, one or more of the scAAV vectors disclosed herein, such as for example pharmaceutical formulations of the vectors intended for administration to a mammal through suitable means, such as, by intramuscular, intravenous, intra-articular, or direct injection to one or more cells, tissues, or organs of a selected mammal, including for example, the subsynovium, a joint capsule, tendon, ligament, cartilage, periarticular muscle, or joint space.

Typically, such compositions may be formulated with pharmaceutically-acceptable excipients as described hereinbelow, and may comprise one or more liposomes, lipids, lipid complexes, microspheres or nanoparticle formulations to facilitate administration to the selected organs, tissues, and cells for which therapy is desired.

Kits comprising one or more of the disclosed scAAV vectors, virions, viral particles, transformed host cells or pharmaceutical compositions comprising such; and (ii) instructions for using the kit in a therapeutic, diagnostic, or clinical embodiment also represent preferred aspects of the present disclosure. Such kits may further comprise one or more reagents, restriction enzymes, peptides, therapeutics, pharmaceutical compounds, or means for delivery of the compositions to host cells, or to an animal, such as syringes, injectables, and the like.

Such kits may be therapeutic kits for treating, preventing, or ameliorating the symptoms of particular joint disease, joint deficiency, joint dysfunction, and/or joint injury, and will typically comprise one or more of the modified scAAV vector constructs, expression systems, virion particles, or therapeutic compositions described herein, and instructions for using the kit. Such kits may also be used in large-scale production methodologies to produce large quantities of the viral vectors.

Another important aspect of the present invention concerns methods of use of the disclosed scAAV vectors, virions, expression systems, compositions, and host cells described herein in the preparation of medicaments for preventing, treating or ameliorating the symptoms of various diseases, dysfunctions, or deficiencies in an animal, such as a vertebrate mammal. Such methods generally involve administration to a mammal, or human in need thereof, one or more of the disclosed vectors, virions, viral particles, host cells, compositions, or pluralities thereof, in an amount and for a time sufficient to prevent, treat, or lessen the symptoms of such a disease, dysfunction, or deficiency in the affected animal. The methods may also encompass prophylactic treatment of animals suspected of having such conditions, or administration of such compositions to those animals at risk for developing such conditions either following diagnosis, or prior to the onset of symptoms.

The invention further provides compositions and methods for treating or ameliorating IL-1Ra polypeptide deficiency in a mammal, or reducing the severity or extent of IL-1Ra polypeptide deficiency in a human. The invention also provides compositions and methods for treating or ameliorating the symptoms of one or more articular disorders, diseases, or dysfunctions, for which direct articular delivery of IL-1Ra polypeptide results in intra-articular expression of biologically-active IL-1Ra polypeptide in an amount and for a time sufficient to treat or ameliorate such conditions.

In a general sense, the method involves administration of one or more scAAV-based genetic construct(s) that comprise a polynucleotide sequence comprising at least one nucleic acid segment that encodes an IL-1Ra peptide or polypeptide to a selected mammalian recipient, in an amount and for a period of time sufficient to treat or ameliorate the deficiency in the animal suspected of suffering from such an articular disease, dysfunction, trauma, injury, and/or disorder.

The invention also provides scAAV virions, and pluralities of double-stranded scAAV vectors, as well as pluralities of viral particles that comprise one of more such double-stranded scAAV vectors. In certain therapeutic embodiments, and in methods involving the compositions for use in the preparation of a medicament for treating or preventing such disorders, the vectors, virions, or viral particles of the present invention are preferably formulated in one or more pharmaceutically-acceptable vehicles, diluents, buffers, or such like that are biocompatible or biologically-acceptable when administered to one or more joints, articular sites, or periarticular spaces within the body of a mammal in need thereof.

The present invention provides a double-stranded, self-complimentary adeno-associated viral vector comprising a polynucleotide that comprises a nucleic acid segment encoding an IL-1Ra polypeptide (including for example, but not limited to, mammalian IL-1Ra polypeptides including those of human, primate, murine, porcine, bovine, ovine, feline, canine, equine, epine, caprine, or lupine origin) operably linked to a promoter capable of expressing the segment in a host cell comprising the vector. In exemplary embodiments, the expressed polypeptide activates the central melanocortin pathway in cells of a mammal that expresses the vector.

In illustrative embodiments, the invention provides an scAAV vector that comprises at least a first nucleic acid that encodes a polypeptide that is at least 85% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:19, SEQ ID NO:20, or SEQ ID NO:21.

Preferably, the invention provides a double-stranded scAAV vector, and compositions comprising it, wherein the scAAV vector comprises at least a first nucleic acid segment that encodes a biologically-active IL-1Ra polypeptide that is at least 90% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:19, SEQ ID NO:20, or SEQ ID NO:21.

More preferably, the invention provides an scAAV vector, and compositions comprising it, wherein the nucleic acid segment encodes a biologically-active IL-1Ra polypeptide that is at least 95% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:19, SEQ ID NO:20, or SEQ ID NO:21.

More preferably still, the invention provides an scAAV vector, and compositions comprising it, wherein the nucleic acid segment comprised within the vector, encodes a biologically-active IL-1Ra polypeptide that is at least 98% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:19, SEQ ID NO:20, or SEQ ID NO:21.

In exemplary embodiments, the invention provides an scAAV vector, and compositions comprising it, in which the nucleic acid segment encodes a biologically-active IL-1Ra polypeptide that comprises, consists essentially of, or consists of the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO: 19, SEQ ID NO:20, or SEQ ID NO:21.

Preferably, the invention provides an scAAV vector (as well as virions, virus particles, and compositions comprising it), wherein the nucleic acid segment comprises, consists essentially of, or consists of, a nucleotide sequence that is at least 80% homologous to the nucleotide sequence of any one of SEQ ID NO:1, SEQ ID NO:3, SEQ ID NO:5, SEQ ID NO:7, SEQ ID NO:9, SEQ ID NO:1, SEQ ID NO:13, SEQ ID NO:15, or SEQ ID NO:17.

More preferably, the invention provides an scAAV vector (as well as virions, virus particles, and compositions comprising it) wherein the nucleic acid segment comprises, consists essentially of, or consists of, a nucleotide sequence that is at least 85% homologous to the sequence of any one of SEQ ID NO: 1, SEQ ID NO:3, SEQ ID NO:5, SEQ ID NO:7, SEQ ID NO:9, SEQ ID NO: 1, SEQ ID NO: 13, SEQ ID NO: 15, or SEQ ID NO:17.

Still more preferably, the invention provides an scAAV vector (as well as virions, virus particles, and compositions comprising it), wherein the nucleic acid segment comprises, consists essentially of, or consists of, a nucleotide sequence that is at least 90% homologous to the sequence of any one of SEQ ID NO:1, SEQ ID NO:3, SEQ ID NO:5, SEQ ID NO:7, SEQ ID NO:9, SEQ ID NO:11, SEQ ID NO: 13, SEQ ID NO:15, or SEQ ID NO:17.

Even more preferably, the invention provides an scAAV vector (as well as virions, virus particles, and compositions comprising it), wherein the nucleic acid segment comprises, consists essentially of, or consists of, a nucleotide sequence that is at least 95% homologous to the sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, or SEQ ID NO:17.

In exemplary embodiments, the invention provides an scAAV vector (as well as virions, virus particles, and compositions comprising it), wherein the nucleic acid segment comprises, consists essentially of, or consists of, the nucleotide sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, or SEQ ID NO:17.

The scAAV viral vectors of the present invention may comprise one or more constitutive, inducible, or tissue-specific promoters, including for example, but not limited to, one or more promoters selected from the group consisting of a CMV promoter, a P-actin promoter, a hybrid CMV enhancer/β-actin promoter, an EF1 promoter, an U1a promoter an U1b promoter, or a promoter that is specifically, or preferably expressed in one or more joints, articular sites, and/or one or more periarticular spaces, including, for example, tissue-specific or joint-specific mammalian promoters.

In certain embodiments, the vectors of the present invention may also optionally comprise one or more enhancers, enhancer elements, or regulatory sequences operably linked to the nucleic acid segment the comprises the therapeutic gene. Examples of such enhancer elements include, but are not limited to, the CMV enhancer.

Likewise, in some embodiments, the vectors of the present invention may also optionally comprise one or more post-transcriptional regulatory elements, such as for example, the woodchuck hepatitis virus post-transcription regulatory element.

The invention also provides an adeno-associated viral vector comprising a polynucleotide that comprises a nucleic acid segment that is at least 90% homologous to the sequence of any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, or SEQ ID NO:17, wherein such a nucleic acid segment encodes a biologically-active IL-1Ra polypeptide operably linked to a promoter capable of expressing such a segment in a host cell that comprises the vector.

Likewise, the invention also provides an adeno-associated viral vector comprising a polynucleotide that comprises a nucleic acid segment that is at least 92% homologous to the sequence of any one of SEQ ID NO:1, SEQ ID NO:3, SEQ ID NO:5, SEQ ID NO:7, SEQ ID NO:9, SEQ ID NO: 1, SEQ ID NO:13, SEQ ID NO:15, or SEQ ID NO:17, wherein such a nucleic acid segment encodes a biologically-active IL-1Ra polypeptide operably linked to a promoter capable of expressing such a segment in a host cell that comprises the vector.

More preferably, the invention discloses and claims adeno-associated viral vectors that comprise at least a first polynucleotide that comprises a nucleic acid segment that is at least 94% homologous to the sequence of any one of SEQ ID NO: 1, SEQ ID NO:3, SEQ ID NO:5, SEQ ID NO:7, SEQ ID NO:9, SEQ ID NO:11, SEQ ID NO:13, SEQ ID NO:15, or SEQ ID NO:17, wherein such a nucleic acid segment encodes a biologically-active IL-1Ra polypeptide operably linked to a promoter capable of expressing such a segment in a host cell that comprises the vector.

More preferably still, the invention discloses and claims adeno-associated viral vectors that comprise at least a first polynucleotide that comprises a nucleic acid segment that is at least 96% homologous to the sequence of any one of SEQ ID NO: 1, SEQ ID NO:3, SEQ ID NO:5, SEQ ID NO:7, SEQ ID NO:9, SEQ ID NO:11, SEQ ID NO:13, SEQ ID NO: 15, or SEQ ID NO: 17, wherein such a nucleic acid segment encodes a biologically-active IL-1Ra polypeptide operably linked to a promoter capable of expressing such a segment in a host cell that comprises the vector.

Even more preferably, the invention provides adeno-associated viral vectors that comprise at least a first polynucleotide that comprises a nucleic acid segment that is at least 98% homologous to the sequence of any one of SEQ ID NO: 1, SEQ ID NO:3, SEQ ID NO:5, SEQ ID NO:7, SEQ ID NO:9, SEQ ID NO:11, SEQ ID NO:13, SEQ ID NO:15, or SEQ ID NO: 17, wherein such a nucleic acid segment encodes a biologically-active IL-1Ra polypeptide operably linked to a promoter capable of expressing such a segment in a host cell that comprises the vector.

In certain illustrative embodiments, the invention provides adeno-associated viral vectors that comprise at least a first polynucleotide that comprises a nucleic acid segment that comprises, consists essentially of, or consists of a sequence that is at least 99% homologous to the sequence of any one of SEQ ID NO: 1, SEQ ID NO:3, SEQ ID NO:5, SEQ ID NO:7, SEQ ID NO:9, SEQ ID NO: 1, SEQ ID NO:13, SEQ ID NO:15, or SEQ ID NO:17, wherein such a nucleic acid segment encodes a biologically-active IL-1Ra polypeptide operably linked to a promoter capable of expressing such a segment in a host cell that comprises the vector.

In additional embodiments, the invention also provides an adeno-associated viral vector comprising at least a first polynucleotide that comprises a nucleic acid segment that encodes a mammalian IL-1Ra polypeptide that is at least 91% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ, SEQ ID NO: 19, SEQ ID NO:20, or SEQ ID NO:21, operably linked to a promoter capable of expressing the nucleic acid segment in a host cell that comprises the vector, wherein the expression of the nucleic acid segment results in the production of substantially biologically-active IL-1Ra peptide or polypeptide in the cells or tissues of a mammal that expresses such a vector.

Likewise, the invention provides an adeno-associated viral vector comprising at least a first polynucleotide that comprises a nucleic acid segment that encodes a mammalian IL-1Ra polypeptide that is at least 93% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ, SEQ ID NO:19, SEQ ID NO:20, or SEQ ID NO:21, operably linked to a promoter capable of expressing the nucleic acid segment in a host cell that comprises the vector, wherein the expression of the nucleic acid segment results in the production of substantially biologically-active IL-1Ra peptide or polypeptide in the cells or tissues of a mammal that expresses such a vector.

More preferably, the invention also provides an adeno-associated viral vector comprising at least a first polynucleotide that comprises a nucleic acid segment that encodes a mammalian IL-1Ra polypeptide that is at least 95% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ, SEQ ID NO:19, SEQ ID NO:20, or SEQ ID NO:21, operably linked to a promoter capable of expressing the nucleic acid segment in a host cell that comprises the vector, wherein the expression of the nucleic acid segment results in the production of substantially biologically-active IL-1Ra peptide or polypeptide in the cells or tissues of a mammal that expresses such a vector.

Even more preferably, the invention provides an adeno-associated viral vector comprising at least a first polynucleotide that comprises a nucleic acid segment that encodes a mammalian IL-1Ra polypeptide that is at least 97% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ, SEQ ID NO:19, SEQ ID NO:20, or SEQ ID NO:21, operably linked to a promoter capable of expressing the nucleic acid segment in a host cell that comprises the vector, wherein the expression of the nucleic acid segment results in the production of substantially biologically-active IL-1Ra peptide or polypeptide in the cells or tissues of a mammal that expresses such a vector.

Likewise, the invention also provides an adeno-associated viral vector comprising at least a first polynucleotide that comprises a nucleic acid segment that encodes a mammalian IL-1Ra polypeptide that is at least 99% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ, SEQ ID NO:19, SEQ ID NO:20, or SEQ ID NO:21, operably linked to a promoter capable of expressing the nucleic acid segment in a host cell that comprises the vector, wherein the expression of the nucleic acid segment results in the production of substantially biologically-active IL-1Ra peptide or polypeptide in the cells or tissues of a mammal that expresses such a vector.

In certain illustrative embodiments, the invention provides adeno-associated viral vectors that comprise at least a first polynucleotide that comprises a nucleic acid segment that encodes a mammalian IL-1Ra polypeptide that comprises, consists essentially of, or consists of the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ, SEQ ID NO:19, SEQ ID NO:20, or SEQ ID NO:21.

The invention also provides recombinant adeno-associated virus virions, viral particles, and pluralities or populations of such virions and/or viral particles that comprise, or substantially all of which comprise one or more of the IL-1Ra-encoding, scAAV vectors described herein. Likewise, the invention also discloses and claims compositions that comprise one or more of the polynucleotides, polypeptides, vectors, virus particles, and/or virions described herein. Such compositions may further optionally comprise at least one or more physiologically-acceptable buffers, diluents, or pharmaceutically-acceptable excipients, carriers, or vehicles, as described herein.

Example of these include, but are not limited to, compositions that further comprise one or more buffers, liposomes, lipids, lipid complexes, microspheres, microparticles, nanospheres, nanocapsules, or nanoparticles. Such compositions may be formulated for administration to one or more tissues, organs, cells, or systems of a mammal, with formulations for administration to veterinary animals, and primates, including humans, being particularly preferred.

In embodiments where administration of pharmaceutical formulations of the invention to a mammal is contemplated, such compositions may be formulated for systemic, intramuscular, intra-articular, or by direct injection or administration to one or more cells or tissues of the recipient mammal using one or more of the conventional methodologies for scAAV administration, as known and routinely used by one of skill in these arts. In methods employing such compositions, they may be formulated by any conventional method, such that the vectors, virions, or viral particles comprised within such compositions are introduced into suitable cell(s), organ(s), or tissue(s) by suitable means, including for example, by infection, transfection, or by direct injection.

The invention also provides diagnostic and therapeutic kits for diagnosing, preventing, treating, and/or ameliorating the symptoms of one or more diseases, disorders, and/or dysfunctions caused by a lack of functional IL-1Ra polypeptide activity in a mammal, or by a deficiency in the level of IL-1RA polypeptide, or a deficiency in the activity of native IL-IRA protein in one or more articular regions of the mammalian body. Such kits typically will comprise: (i) one or more of the scAAV-IL-1RA vectors, virions viral particles, or compositions as disclosed herein; and (ii) instructions for using the kit in the administration of such composition(s) either systemically or locally e.g., to one or more articular, intraarticular, or extra-articular regions of a mammalian body in general, and to a human body in particular.

The invention also provides uses of the disclosed compositions in therapy, and particularly in therapeutic methods for treating articular diseases, disorders and/or dysfunctions in a mammal. These methods generally comprise at least the step of introducing into a cell, or to a plurality of cells or tissue of the mammal, a therapeutically-effective amount of one or more of the disclosed scAAV-IL-1RA vectors (as well as virions, pluralities of virus particles, or compositions comprising them), for a time effective to treat the intended condition, or to ameliorate the symptoms of the disease or disorder. Preferably, the mammal is a human under the care of a physician or suitable health care professional, or in the case of non-human animals, the mammal is preferably under the care of a veterinarian.

Also provided is a method for treating or ameliorating the symptoms of an IL-1Ra polypeptide deficiency condition in a mammal. The method generally involves at least the step of administering to such a one or more of the scAAV-IL-1RA vectors, virions viral particles, or compositions as disclosed herein, in an amount and for a time sufficient to treat or ameliorate the symptoms of such a deficiency in the mammal.

The invention also provides a method for providing to a mammal in need thereof, a therapeutically-effective amount of an IL-1Ra peptide or polypeptide. Such method generally involves directly introducing into suitable cell(s), organ(s), and/or tissue(s) of the mammal, a biologically-effective amount of an IL-1Ra peptide or polypeptide for a time effective to provide the mammal with a therapeutically-effective amount of the IL-1Ra polypeptide. Such methodologies may be involved directly using an in vivo or in situ treatment regimen, where the scAAV-IL-1RA vectors or IL-IRA compositions are directly introduced into the living organism itself, or alternatively, the process may involve treating one or more cell(s), tissue(s), and/or organ(s) of the animal in an ex vivo, or in vitro, fashion, and then reintroducing such treated cells, tissues, or organ back into the body of the animal in need of the treatment.

2.1 scAAV-IL-1RA Vector Compositions

In one embodiment, the invention provides an scAAV vector comprising a polypeptide that comprises at least a first nucleic acid segment that encodes a mammalian IL-1Ra peptide or polypeptide, and in particular, a biologically-active interleukin-1 receptor antagonist (IL-1Ra) polypeptide, or biologically-active fragment thereof, operably linked to at least a first promoter capable of expressing the nucleic acid segment in a suitable host cell transformed with such a vector. In preferred embodiments, the nucleic acid segment encodes a mammalian, and in particular, a human IL-1Ra polypeptide, such as for example, biologically-active contiguous amino acid sequences that comprise, consist essentially of, or consist of, one or more of the polypeptide sequences as disclosed in any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:19, SEQ ID NO:20, or SEQ ID NO:21.

In addition to therapeutic polynucleotides and polypeptides of human origin, the invention also encompasses treatment modalities involving the use of one or more other mammalian IL-1Ra-encoding nucleotides or IL-1Ra peptides, proteins, or polypeptides, as may be desirable in the treatment of humans, or other mammals (such as for example, in veterinary medicine therapies), and as such, the scAAV vectors may comprise IL-1Ra-encoding sequences that are derived in whole or in part of native, mutated, or synthetically-modified sequences, including those of human, primate, murine, porcine, feline, canine, bovine, ovine, equine, epine, caprine, or lupine origin. In an example presented herein as an illustrative embodiment of the practice of the invention, the scAAV-IL-1Ra constructs comprise at least a first genetic sequence that encodes a human IL-1Ra peptide, polypeptide, or protein, to provide therapeutic levels of the selected protein, e.g., IL-1Ra, to the transfected cells.

Alternatively, the therapeutic constructs of the invention may encompass nucleic acid segments that encode modified IL-1Ra polypeptides obtained from any mammalian origin, and engineered by the hand of man to produce more desirable properties or characteristics. For example, nucleic acids, peptides, proteins, and polypeptides of murine, primate, ovine, porcine, bovine, equine, epine, caprine, canine, feline, avian, amphibian, and/or lupine origin, may be used in their native or unmodified form, but also may be modified or site-specifically mutagenized, and/or otherwise genetically modified to be expressed in human cells such that their IL-1Ra biological activity is retained, increased, or prolonged.

Preferred AAV vector backbones for the practice of the present invention include, but are not limited to, AAV serotype 1 (AAV1), AAV serotype 2 (AAV2), AAV serotype 3 (AAV3), AAV serotype 4 (AAV4), AAV serotype 5 (AAV5), or AAV serotype 6 (AAV6) vectors, or derivates thereof.

The polynucleotides comprised in the vectors and viral particles of the present invention preferably comprise at least a first constitutive or inducible promoter operably linked to the nucleic acid segments disclosed herein. Such promoters may be homologous or heterologous promoters, and may be operatively positioned upstream of the nucleic acid segment encoding the therapeutic polypeptide of interest, such that the expression of the segment is under the control of the promoter. The construct may comprise a single promoter, or alternatively, two or more promoters may be used to facilitate expression of the therapeutic gene sequence.

Exemplary promoters useful in the practice of the invention include, but are in no way limited to, those promoter sequences that are operable in mammalian, and in particular, human host cells, tissues, and organs, such as for example, a CMV promoter, a β-actin promoter, a hybrid CMV promoter, a hybrid β-actin promoter, an EF1 promoter, a U1a promoter, a U1b promoter, a Tet-inducible promoter and a VP16-LexA promoter being particularly useful in the practice of the invention. In particular applications of the disclosed invention, the inventors contemplate that a polynucleotide encoding an IL-1Ra peptide or polypeptide may be placed under the control of a suitable promoter [e.g., a chicken M-actin (CBA) promoter] and used to produce therapeutically effective levels of the encoded IL-1Ra polypeptide when suitable host cells were transformed with the genetic construct.

The polynucleotides comprised in the vectors and viral particles of the present invention may also further optionally comprise one or more native, synthetic, homologous, heterologous, or hybrid enhancer or 5′ regulatory elements, for example, a CMV enhancer, a synthetic enhancer, or an organ- or tissue-specific enhancer operably linked to the therapeutic IL-1Ra polypeptide-encoding nucleic acid segments disclosed herein.

The polynucleotides and nucleic acid segments comprised within the vectors and viral particles of the present invention may also further optionally comprise one or more intron sequences. Such sequences may be from native IL-1Ra genes, or from other non-related genes.

The IL-1Ra-encoding polynucleotides comprised in the vectors and viral particles of the present invention may also further optionally comprise one or more native, synthetic, homologous, heterologous, or hybrid post-transcriptional or 3′ regulatory elements operably positioned relative to the therapeutic polypeptide-encoding nucleic acid segments disclosed herein to provide greater expression, stability, or translation of the encoded polypeptides. One such example is the woodchuck hepatitis virus post-transcriptional regulatory element (WPRE), operably positioned downstream of the therapeutic gene(s) of interest.

In illustrative embodiments, the invention concerns administration of one or more biologically-active IL-1Ra peptides or polypeptides that comprises, consists essentially of, or consists of, an at least 20, at least 30, at least 40, at least 50, at least 60, at least 70, at least 80, at least 90, at least 100, at least 110, at least 120, at least 130, at least 140, or at least 150, 160, 170, 180, 190, or longer contiguous amino acid sequence from one or more of the polypeptide sequences disclosed hereinbelow and particularly those polypeptides as recited in any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:19, SEQ ID NO:20, or SEQ ID NO:21.

Likewise, in additional illustrative embodiments, the invention concerns administration of one or more biologically-active IL-1Ra polypeptides that are encoded by a nucleic acid segment that comprises, consists essentially of, or consists of, at least 30, at least 60, at least 90, at least 120, at least 150, at least 180, at least 210, at least 240, at least 270, at least 300, at least 330, at least 360, at least 390, at least 420, at least 450, at least 480, at least 510, at least 540, at least 570, or at least 600, 700, 800, or 900, or longer contiguous nucleic acid residues, up to and including substantially full-length, and full-length sequences from the DNA sequences disclosed hereinbelow and particularly nucleic acid segments that comprise, consist essentially of, or consist of one or more of the contiguous nucleotide sequences as recited in any one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, and SEQ ID NO:17.

Exemplary adeno-associated viral vector constructs and polynucleotides of the present invention include those that comprise, consist essentially of, or consist of at least a first nucleic acid segment that encodes an IL-1Ra peptide or polypeptide that is at least about 75%, at least about 80%, at least about 85%, at least about 90%, at least about 95%, at least about 96%, at least about 97%, at least about 98%, or at least about 99% identical to the sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:19, SEQ ID NO:20, or SEQ ID NO:21, wherein the peptide or polypeptide has IL-1Ra activity when administered to, and expressed in, a suitable mammalian cell, tissue, or organ.

Exemplary polynucleotides of the present invention also include those sequences that comprise, consist essentially of, or consist of at least a first nucleic acid segment that encodes a polypeptide that is at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:19, SEQ ID NO:20, or SEQ ID NO:21, wherein the peptide or polypeptide encoded by the nucleic acid segment has IL-1Ra activity when expressed in a suitable mammalian cell, tissue or organ.

Particularly preferred adeno-associated viral vector constructs and polynucleotides of the present invention include those that comprise, consist essentially of, or consist of at least a first nucleic acid segment that is at least about 75%, at least about 80%, at least about 85%, at least about 90%, at least about 95%, at least about 96%, at least about 97%, at least about 98%, or at least about 99% identical to the sequence of any one of SEQ ID NO:1, SEQ ID NO:3, SEQ ID NO:5, SEQ ID NO:7, SEQ ID NO:9, SEQ ID NO: 1, SEQ ID NO:13, SEQ ID NO:15, and SEQ ID NO:17, wherein the segment encodes a peptide or polypeptide that has IL-1Ra activity when administered to, and expressed in, a suitable mammalian cell.

Exemplary polynucleotides of the present invention also include those sequences that comprise, consist essentially of, or consist of at least a first nucleic acid segment that is at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of any one of SEQ ID NO:1, SEQ ID NO:3, SEQ ID NO:5, SEQ ID NO:7, SEQ ID NO:9, SEQ ID NO:1, SEQ ID NO:13, SEQ ID NO:15, or SEQ ID NO:17, wherein the peptide or polypeptide encoded by the nucleic acid segment has IL-1Ra activity when expressed in a suitable mammalian cell, tissue, or organ.

### 3. BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1 shows infection of primary human synovial fibroblasts with self-complementary AAV (scAAV) encoding the cDNA for human interleukin-1 receptor antagonist (IL-1Ra; scAAV.IL-1Ra) results in high level expression of the transgene. Self-complementary (sc) AAV vector encoding IL-1Ra (scAAV.IL-1Ra) was produced by inserting an expression cassette (containing the cytomegalovirus promoter/enhancer followed by the complete cDNA for human IL-1Ra and two poly A tail regions) between the two inverted terminal repeat regions of AAV2 of the pUF5 plasmid. The AAV viral vector construct results in a recombinant AAV genome that is approximately half (˜2.2 kb) of the wild type genome (4.6 kb).

Following co-transfection of the scAAV-IL-1Ra plasmid with a helper packaging plasmid containing the AAV2 capsid protein into 293 cells, cell lysates were fractionated over cesium chloride gradients. Fractions from the gradient were tested for infectious virus by infection of 293 cells. Active fractions were pooled and dialyzed. To test the function of the recombinant vector, cultures of primary synovial fibroblast cells were plated and allowed to grow to confluence. The cells were then incubated with increasing amounts of the AAV.IL-1Ra virus. At 24-hr intervals post-infection, the culture media were removed and stored. The cells were washed with saline and the media replaced. IL-1Ra levels in culture supernatants from days 1, 3, and 7 were determined using ELISA measurements. Each viral dose was tested in triplicate and the bars represent the mean ±one S.D. As shown in the graph the onset of transgenic expression was quite rapid, occurring in 24 hrs and was dose-dependent. In these cells which were rapidly dividing, the episomal AAV genome did not persist, and transgene expression diminished over the 7 day experiment.

FIG. 2A and FIG. 2B show scAAV.GFP or scAAV.IL-1Ra packaged in AAV capsid proteins from serotypes 2, 3, 5, 7 and 8 as indicated. Approximately 10⁴ particles per cell were used to infect monolayer cultures of capsular fibroblasts isolated from the joints of a horse. Expression of the GFP transgene was observed by fluorescence microscopy. Expression of the IL-1Ra transgene was measured by ELISA of the conditioned media at days 3, 7, 14 and 21 post-infection. As shown in FIG. 2A serotypes 2, 3 and 5 provided greater intensity of GFP expression than type 8. In agreement with these findings IL-1Ra synthesis from cells infected with serotypes 2 and 5 was greater than that from cells infected with serotypes 7 and 8 (FIG. 2B).

FIG. 3A and FIG. 3B show intra-articular expression of scAAV.IL-1Ra following direct injection into normal and inflamed rabbit knee joints. Ten rabbits were initially injected in both knees with 5×10⁴ HIG-82-IL-1 cells which stimulates an immediate, persistent inflammatory state. Three days later, 5×10¹¹ particles of scAAV.IL-1Ra were injected into both knees of 5 of the rabbits with inflamed knees and 5 normal rabbits. An equivalent volume of saline was injected into the remaining 5 inflamed rabbits and an additional 5 normal rabbits. At periodic intervals after the viral injection, the knees of all rabbits were lavaged with saline and the IL-1Ra content in recovered fluids measured by ELISA (FIG. 3A).

Infiltrating leukocytes in lavage fluids recovered at days 3 and 7 for each group were quantitated using a hemocytometer (FIG. 3B). Values shown represent the mean ±one S.D. These data demonstrate that the scAAV vector enables rapid onset to transgene expression following intra-articular injection at levels that are therapeutically relevant.

FIG. 4A and FIG. 4B shows the results of studies in which rats were injected intra-articularly with scAAV containing the cDNA for green fluorescent protein (GFP). In this case, the recombinant vector DNA was packaged using AAV capsid proteins from serotype 8. Five days post-injection, the animals were killed, and the joints were harvested. The intact joint tissues were examined under fluorescent light, and the entire region immediately surrounding the joint capsule was noted to fluoresce green. Dissection of the tissues demonstrated that the majority of GFP expression occurred in the musculature immediately surrounding the joint capsule (FIG. 4B). Further dissection showed significant transgenic expression in the fibroblastic cells of the joint capsule (FIG. 4A). Although the images are of serotype 8, similar results were seen with serotype 5.

FIG. 5 shows transgene expression following intra-articular injection of scAAV.IL-1Ra into the knees of rat. Several groups of rats were injected in the joint space with 3×10⁹ viral particles of scAAV.IL-1Ra packaged in serotypes, 2, 5, 7, or 8. Five days post-injection the animals were sacrificed. The knees were harvested, dissected to expose the articular surfaces and cultured ex vivo for 24 hrs. Expression of the IL-1Ra transgene was measured by ELISA of the conditioned media. Levels of expression are shown on the vertical axis as pg/ml.

FIG. 6 shows self complementary AAV (scAAV) provides improved transduction of rabbit capsular fibroblasts. As shown here, the scAAV-GVP enabled significantly greater transduction of the rabbit cells. Expression was noted to diminish somewhat by day 7. For each plot levels of fluorescence are represented on the horizontal axes and cell size on the vertical axes. Uninfected cells were used to establish levels of background fluorescence of the cells and were used as negative controls as indicated.

FIG. 7 shows self complementary AAV (scAAV) provides improved transduction of rabbit capsular fibroblasts. As seen here, the single stranded vector provides very limited modification of the fibroblastic cells while the scAAV enables rapid onset of expression.

FIG. 8A, FIG. 8B, and FIG. 8C show scAAV-mediated gene transfer into equine joints enables high level expression of therapeutic transgene products. Approximately 2×10¹¹ viral genomes were injected into both front carpal joints (knees) and metacarpalphalangeal joints (ankles) of several horses (FIG. 8A.). At periodic intervals, synovial fluid was aspirated from each of the joints (FIG. 8B) and analyzed for the presence of human IL-1Ra using species specific ELISA (FIG. 8C).

### 4. DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

### 4.1 Viral Vector-Based Gene Therapy

Major advances in the field of gene therapy have been achieved by using viruses to deliver therapeutic genetic material. The adeno-associated virus (AAV) has attracted considerable attention as a highly effective viral vector for gene therapy due to its low immunogenicity and ability to effectively transduce non-dividing cells. AAV has been shown to infect a variety of cell and tissue types, and significant progress has been made over the last decade to adapt this viral system for use in human gene therapy.

In its normal “wild type” form, recombinant AAV (rAAV) DNA is packaged into the viral capsid as a single stranded molecule about 4600 nucleotides in length. Following infection of the cell by the virus, the molecular machinery of the cell converts the single DNA strand into a double-stranded form. Only the double stranded DNA form is useful to the proteins of the cell that transcribe the contained gene or genes into RNA.

AAV has many properties that favor its use as a gene delivery vehicle. 1) The wild type virus is not associated with any pathologic human condition. 2) The recombinant form does not contain native viral coding sequences. 3) Persistent transgenic expression has been observed in many applications.

A unique feature of the AAV virus is its small particle size relative to other well-characterized viral-based gene delivery systems such as recombinant adenovirus, lentivirus and herpes simplex virus. The small particle size of the virus enables it to penetrate and traverse the dense matrix of the capsular tissues to transduce resident capsular fibroblasts as well as periarticular muscle fibers and connective tissues.

Historically, intra-articular gene transfer has involved delivery of genetic material to cells of the synovial lining, primarily type B synovial fibroblasts. In recent studies, however, it has been shown that these cells typically do not persist within the joint tissues for longer than a few weeks. These data support a model whereby synovial fibroblasts, due to their proximity to the articulating surfaces of the bones, are physically sloughed off and turn over every few days or weeks. Because of this, most of these cells are unable to support long-term expression of an exogenous therapeutic transgene. This is particularly relevant when devising gene therapy methods to address chronic, lifelong joint diseases. In previous experiments using adenovirus, herpes simplex virus and lentiviral vectors, the vast majority (>95%) of gene transfer was found in the cells of the synovial lining. Following intra-articular injection, significant gene transfer to sites beyond the joint capsule has not been previously reported.

### 4.2 AAV Serotypes

There exist a large number of naturally occurring strains of AAV with divergent DNA sequences and capsid proteins that are of distinct serotypes and have different tropisms. The scAAV vector that has been exploited by the present invention contains terminal repeat regions of the genome of AAV serotype 2. However, similar types of viral schemes can be used to generate scAAV for intra-articular gene transfer from the genomes of other serotypes.

It has been shown that scAAV comprised of terminal repeats from one serotype when packaged into the viral capsid of other serotypes remains functional for intra-articular gene transfer and in some cases can improve the efficiency of gene transfer and ensuing transgenic expression. For example, scAAV type 2 packaged into capsid protein of serotypes 2, 3, and 5 efficiently transduce fibroblastic cells from joint capsular tissues well in vitro. However, following intra-articular injection scAAV 2 packaged into serotypes 5, 7, and 8 permitted high levels of intra-articular expression, with serotypes 5 and 8 enabling the greatest levels of expression. Thus, the self-complementary AAV vector can be combined with a variety of different capsid serotypes to provide levels of transgenic expression that have been shown to be therapeutically relevant.

### 4.3 Pharmaceutical Compositions

The genetic constructs of the present invention may be prepared in a variety of compositions, and may also be formulated in appropriate pharmaceutical vehicles for administration to human or animal subjects. The scAAV molecules of the present invention and compositions comprising them provide new and useful therapeutics for the treatment, control, and amelioration of symptoms of a variety of disorders, and in particular, articular diseases, disorders, and dysfunctions, including for example osteoarthritis, rheumatoid arthritis, and related disorders.

Moreover, pharmaceutical compositions comprising one or more of the nucleic acid compounds disclosed herein, provide significant advantages over existing conventional therapies—namely, (1) their reduced side effects, (2) their increased efficacy for prolonged periods of time, (3) their ability to increase patient compliance due to their ability to provide therapeutic effects following as little as a single administration of the selected therapeutic scAAV composition to affected individuals. Exemplary pharmaceutical compositions and methods for their administration are discussed in significant detail hereinbelow.

The invention also provides compositions comprising one or more of the disclosed scAAV vectors, expression systems, virions, viral particles; or mammalian cells. As described hereinbelow, such compositions may further comprise a pharmaceutical excipient, buffer, or diluent, and may be formulated for administration to an animal, and particularly a human being. Such compositions may further optionally comprise a liposome, a lipid, a lipid complex, a microsphere, a microparticle, a nanosphere, or a nanoparticle, or may be otherwise formulated for administration to the cells, tissues, organs, or body of a mammal in need thereof.

Such compositions may be formulated for use in a variety of therapies, such as for example, in the amelioration, prevention, and/or treatment of conditions such as IL-1Ra peptide or polypeptide deficiency, and/or overexpression of interleukin-1 receptor (IL-1R) peptides or polypeptides in one or more cells or tissues of the mammal into which the disclosed vector compositions are introduced.

In certain embodiments, the present invention concerns formulation of one or more scAAV-based compositions disclosed herein in pharmaceutically acceptable solutions for administration to a cell or an animal, either alone or in combination with one or more other modalities of therapy, and in particular, for therapy of human articular diseases, disorders, injuries, physical trauma, and/or dysfunctions.

The pharmaceutical forms of the scAAV-based vector compositions suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active AAV vector-delivered therapeutic polypeptide-encoding DNA fragments in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The phrase “pharmaceutically-acceptable” refers to molecular entities and compositions that do not produce an allergic or similar untoward reaction when administered to a human, and in particular, when administered to one or more cells and/or tissues of a mammal, and of a human in particular. The amount of an scAAV vector composition and time of administration of such compositions will be within the purview of the skilled artisan having benefit of the present teachings. It is likely, however, that the administration of therapeutically-effective amounts of the disclosed scAAV-based IL-1Ra therapeutic compositions may be achieved by a single administration, such as for example, a single injection of sufficient numbers of viral particles to provide therapeutic benefit to the patient undergoing such treatment. Alternatively, in some circumstances, it may be desirable to provide multiple, or successive administrations of the scAAV vector compositions, either over a relatively short, or a relatively prolonged period of time, as may be determined by the medical practitioner overseeing the administration of such compositions.

For example, the number of infectious particles administered to a mammal may be on the order of about 10⁷, 10⁸, 10⁹, 10¹⁰, 10¹¹, 10¹², 10¹³, or even higher, infectious particles/ml given either as a single dose, or divided into two or more administrations as may be required to achieve therapy of the particular disease or disorder being treated. In fact, in certain embodiments, it may be desirable to administer two or more different AAV vector compositions, either alone, or in combination with one or more other therapeutic drugs to achieve the desired effects of a particular therapy regimen.

### 4.4 Expression Vectors

The present invention contemplates a variety of scAAV-based expression systems and vector constructs. In one embodiment the preferred scAAV expression vectors comprise at least a first nucleic acid segment that encodes a biologically-active, mammalian IL-1Ra peptide, protein, or polypeptide.

As used herein, the term “operatively linked” means that a promoter is connected to a functional RNA in such a way that the transcription of that functional RNA is controlled and regulated by that promoter. Means for operatively linking a promoter to a functional RNA are well known in the art.

### 4.5 Therapeutic Kits

The invention also encompasses one or more of the modified scAAV vector compositions described herein together with one or more pharmaceutically-acceptable excipients, carriers, diluents, adjuvants, and/or other components, as may be employed in the formulation of particular scAAV vector-delivered formulations, and in the preparation of therapeutic agents for administration to a mammal, and in particularly, to one or more joint or articular sites in a human. In particular, such kits may comprise one or more of the disclosed scAAV vector compositions in combination with instructions for using the viral vector in the treatment of such disorders in a mammal, and may typically further include containers prepared for convenient commercial packaging.

As such, preferred animals for administration of the pharmaceutical compositions disclosed herein include mammals, and particularly humans. Other preferred animals include, but are not limited to, murines, ovines, bovines, equines, porcines, epines, lupines, canines, felines, and non-human primates. The composition may include partially or significantly purified scAAV vector compositions, either alone, or in combination with one or more additional active ingredients, which may be obtained from natural or recombinant sources, or which may be obtainable naturally or either chemically synthesized, or alternatively produced in vitro from recombinant host cells expressing DNA segments encoding such additional active ingredients.

Kits may also be prepared that comprise at least one of the formulations disclosed herein and one or more protocols, instructions, or directions for using the compositions, such as, for example, in the prevention, diagnosis, treatment or amelioration of symptoms of one or more articular diseases, injury, trauma, and/or dysfunction of a joint. The container means for such kits may typically comprise at least one vial, test tube, flask, bottle, syringe, catheter, or other container or delivery means, into which the disclosed therapeutic composition(s) may be placed, and preferably suitably aliquotted. Where a second component is also provided, the kit may also contain a second distinct container means into which this second composition may be placed.

Alternatively, a plurality of compositions may be prepared in a single pharmaceutical composition, and may be packaged in a single container means, such as a vial, flask, syringe, bottle, articular delivery device, catheter, endoscopic instrument, or other suitable single container and/or delivery means. The kits of the present invention will also typically include a means for containing the vial(s) in close confinement for commercial sale, such as, e.g., injection or blow-molded plastic containers into which the desired vial(s) are retained.

### 4.6 The Synovium and Arthritis

The synovium is a tissue that lines all surfaces inside the joint capsule except cartilage.⁹ In healthy joints, it typically exists as a thin lining layer of cells, two to three layers in thickness (˜25 μm), that overlays a subsynovium comprised of adipose, areolar or fibrous connective tissue depending on the anatomical position within the joint. Synovium is highly vascular, and the exchange of soluble molecules between the synovial capillaries and the joint space is the means by which the avascular articular tissues, such as cartilage, are nourished. The majority of cells that populate the synovium can be classified into two cell types, bone marrow-derived macrophage cells (type A synovial cells) and fibroblast-like synoviocytes (type B synovial cells).¹⁰⁻¹³ The type A cells primarily populate the surface of the lining layer, are phagocytic, and can be identified by monoclonal antibodies to macrophage cell surface antigens such as CD14 and CD 11b. The type B cells primarily reside beneath the type A cells in the lining and are responsible for synthesis of hyaluronan, a primary component of synovial fluid. These cells are distinct from type A cells by the presence of Thy1/CD90 and CD29 surface antigens, increased presence of certain adhesion molecules, such as VCAM-1, and their unusually high levels of uridine diphosphoglucose dehydrogenase (UDPGD), a key enzyme in hyaluronan synthesis.^{10-12, 14}

In the two primary arthritic conditions, RA and OA, the physical and biological states of the joint tissues are generally quite different. Although the etiology of RA remains elusive, it is considered to be a systemic autoimmune condition manifested primarily by chronic, symmetric erosive synovitis in peripheral joints. In this disease, the normally thin synovial lining becomes hypercellular and dramatically thickened with numerous villous projections. Increased numbers of macrophage cells migrate from the vasculature, and there is a local proliferation of synovial fibroblasts.^{11,13} These cells are joined by a leukocytic infiltrate comprised of aggregates of CD4+ T cells and plasma cells as well as diffuse mononuclear cells. An infiltrate consisting mainly of neutrophils is also found in the synovial fluid. Constitutive production in the joint of inflammatory cytokines, such as IL-1 and TNF-P, cause the hypertrophied synovium to become activated. Similar to that of a tumor, it acquires an aggressive phenotype and invades articulating tissues, eroding cartilage and subchondral bone. The pathologic process continues throughout the disease course but fluctuates in severity. With time, the generally slow but progressive course of RA can culminate in cartilage and bone destruction, extreme fibrosis and loss of joint function.^{10,11}

OA is characterized primarily by the progressive loss of articular cartilage accompanied by sclerosis of subchondral bone. Synovial changes are less marked in OA than in RA. Although there is disagreement over the degree, duration and relevance of synovial inflammation in OA, the synovium of the OA joint lacks pannus and is not invasive.¹⁵ Synovitis tends to be localized and although it may be symptomatic, is not thought to contribute to the irreversible loss of articular cartilage. There is often evidence of synovial thickening, due to a modest expansion of type B synoviocytes with correspondingly increased deposition of collagen. The extracellular matrix (ECM) of articular cartilage is an intricate network consisting predominately of type II collagen fibers and proteoglycans.

The synthesis, incorporation and degradation of ECM proteins are orchestrated by chondrocytes that populate articular cartilage at low density. Simultaneous synthesis and proteolytic breakdown of certain ECM components by these cells enable much of the cartilage matrix to undergo continual turnover and maintenance. Factors that impair chondrocyte function can disrupt the equilibrium of synthesis and catabolism in favor of cartilage degradation, which over time leads to OA. Although excessive or traumatic joint loading are considered initiating factors, many investigators consider IL-1, synthesized locally by synovial cells and chondrocytes, to drive the progression of the disease.^{16,17} At low levels, IL-1 inhibits proteoglycan and collagen synthesis by chondrocytes,¹⁸ while at higher concentrations, it stimulates increased production of specific tissue degrading enzymes by chondrocytes and synovial fibroblasts. ¹⁹

### 4.7 The Synovium as a Target for Gene Delivery

When designing a local gene delivery strategy to the joint, the synovium is an obvious target tissue for treating both RA and OA. It has a relatively large surface area, and because there is no membrane separating the synovial lining cells from the synovial fluid and joint space, proteins secreted by genetically modified cells are released directly into the synovial fluid and surrounding tissues. Moreover, unlike chondrocytes which reside within a dense extracellular matrix, gene delivery vectors have unimpeded access to the abundant cells within the synovial tissue. Furthermore, following injection into the joint space, synovial fibroblasts in suspension will readily attach and colonize the synovial lining.^{1,20}

A principal motivation for the use of gene-based treatments for chronic joint diseases is the potential to achieve sustained expression and presentation of therapeutic agents at the site of disease. Yet little is known about the capacity of cells within the joint to support the maintenance of a transgene or its expression over time. By far, the primary concentration of research in arthritis gene therapy has been directed toward testing the therapeutic efficacy of various cDNAs. Numerous laboratories have reported beneficial effects in animal models following the intra-articular delivery of genes such as IL-1Ra, soluble IL-1 and TNF receptors, IL-10, viral IL-10, IL-4 etc.,² providing an overwhelming demonstration of the “proof of principle.” Yet in all of these studies, transgene expression in the joint has not been observed to extend beyond two to three weeks. Each report, however, has involved delivery of transgenes from non-homologous species such as human IL-1Ra to the joints of rabbits or rats. This will cause the cells expressing these non-self proteins to be killed by the cellular immune system prior to reaching their normal lifespan. The duration of expression that might be achieved in synovial tissue using transgenes whose products are not seen as “non-self” in the host species has not been determined.

### 4.8 Adeno-Associated Virus

Wild type AAV is a defective human parvovirus that requires helper functions from adenovirus, herpes virus or genotoxic stress for replication.⁴¹ In the absence of helper function, AAV integrates its genome into a specific region of chromosome 19 and remains latent. Vectors based on AAV have several features that favor their use in treating chronic joint disease. Wild-type AAV is non-pathogenic and is not associated with any known disease. Recombinant AAV is engineered so that it encodes no viral proteins, reducing the immunogenicity of the transduced cell in vivo. Because the virus infects a wide variety of dividing and quiescent cells, it can achieve significant levels of cellular transduction following delivery in vivo.⁴² In some tissues, recombinant AAV has been found to integrate into the genome of the target cell as a double stranded provirus enabling stable transduction. Long-term gene expression in vivo (>1 year) mediated by AAV has been reported in several target tissues such as liver, muscle, retina and the central nervous system.⁴³

### 4.9 Animal Models Of Articular Disease

Various models of articular disease are available, including the rabbit knee model system^{34,48} and the rat knee system. The rat knee has been shown to be the smallest mammal that can be accurately and reproducibly injected intra-articularly. This joint safely tolerates liquid volumes of up to 100 μl allowing delivery of cell suspensions and gene transfer vectors. It is also possible to work with up to 50 or more animals simultaneously, and efficiently perform experiments with several groups in parallel. Rats also offer increased availability of specific molecular reagents and antibodies. The greatest advantage of the rat over the rabbit system, however, is the availability of genetically identical animals and immunocompromised strains.

To enable the use of informative marker genes, such as GFP, firefly luciferase and human IL-1Ra, in vivo gene delivery methods using the knee of athymic, nude rats as an animal model is an ideal system. These animals, homozygous for a deletion in the rnu locus, are born without a thymus, and thus, the T-cell portion of their immune system does not function. Because of this, cells expressing foreign epitopes are not killed, but persist indefinitely. Due to its ability to accept “non-self” tissue, this animal may be used as a pseudo-homologous system within which cells expressing GFP, other diagnostic reporter genes, or potentially therapeutic genes will survive for their natural lifespan. Thus, the nude rat provides a unique system within which it is possible to discriminate the specific benefits and limitations of the scAAV gene delivery method with respect to transgenic persistence and to characterize specific cell populations within the synovial lining that may permit long-term expression.

### 4.10 Administration of IL-1RA Compositions to Mammalian Tissues

The invention also provides methods for delivering therapeutically-effective amounts of a biologically-active IL-1Ra polypeptide to a mammal in need thereof. Such methods generally comprise at least the step of providing or administering to such a mammal, one or more of the scAAV-IL-1Ra compositions disclosed herein. For example, the method may involve providing to such a mammal, one or more of the scAAV vectors, virions, viral particles, host cells, or pharmaceutical compositions as described herein. Preferably such providing or such administration will be in an amount and for a time effective to provide a therapeutically-effective amount of one or more of the IL-1Ra peptides or polypeptides disclosed herein to selected cells, tissues, or organs of the mammal, and in particular, therapeutically-effective levels to the cells, tissues, and joints of the mammal. Such methods may include systemic injection(s) of the therapeutic polypeptide composition; however, it is contemplated that a localized, direct administration of the vector composition to one or more articular regions of the mammal will be preferable to systemic administration.

The invention also provides methods of treating, ameliorating the symptoms, and reducing the severity of IL-1Ra deficiency in an animal. These methods generally involve at least the step of providing to an animal in need thereof, one or more of the scAAV vector compositions disclosed herein in an amount and for a time effective to treat the IL-1Ra deficiency or other related dysfunction in the animal. As described above, such methods may involve systemic injection(s) of the therapeuticum, or may even involve direct or indirect administration, injection, or introduction of the therapeutic compositions to particular cells, tissues, or organs of the animal.

In one embodiment, the invention provides a method for treating, preventing, or ameliorating the symptoms of an IL-1Ra protein, peptide, or polypeptide deficiency or dysfunction in a mammal. The method generally involves administering to a mammal in need thereof, one or more of the disclosed scAAV-IL-1Ra vector compositions disclosed herein, in an amount and for a time sufficient to treat, prevent, or ameliorate the symptoms of the IL-1Ra deficiency in the mammal. In preferred embodiments, the mammal is a human that is has, is at risk for developing, or has been diagnosed with one or more diseases, disorders, or dysfunctions that result from the deficiency or lack of one or more IL-1Ra peptides, polypeptides, or proteins normally present in a healthy subject.

In such cases, the compositions of the invention may be administered to the patient in an amount and for a time sufficient to treat or prevent the symptoms of the IL-1Ra deficiency or dysfunction through a single dose, or by administration of a plurality of doses given over a relatively short, or even relatively long period of therapy. The patient may require only one or two administrations of the disclosed scAAV constructs to achieve relatively short-term, relatively medium-term, or even relatively long-term treatment. For example, one or two administrations of the disclosed compositions may provide sufficient therapeutic levels of the IL-1Ra composition for a period of several days, several weeks, or several months. Alternatively, three or four administrations of the disclosed compositions either over a relatively short, or relatively long administration period, may provide sufficient therapeutic levels of the IL-1Ra composition for a period of several weeks, several months, several years, or even tens of years, up to and including the natural lifetime of the treated mammal.

When relatively short-term therapy is warranted, the therapeutic effectiveness of a single administration or of multiple administrations of the disclosed compositions may persist for a period of about 1, 2, 3, 4, 5, 6, 7, 8, 9, or 10 days or more, and even up to an including a period of about 11, 12, 13, 14, 15, 16, 17, 18, 19, or 20 days or more. When relatively medium-term therapy is warranted, the therapeutic effectiveness of a single administration or of multiple administrations of the disclosed compositions may persist for a period of about 1, 2, 3, 4, 5, 6, 7, 8, 9, or 10 weeks or more, and even up to an including a period of about 11, 12, 13, 14, 15, 16, 17, 18, 19, or 20 weeks or more, such as for example, about 21, about 22, about 23, about 24, about 25, about 26, about 27, about 28, about 29, or about 30 weeks or more, and even up to an including a period of about 31, 32, 33, 34, 35, 36, 37, 38, 39, or 40 weeks or more.

Likewise, when relatively long-term therapy is warranted, the therapeutic effectiveness of a single administration or of multiple administrations of the disclosed compositions may persist for a period of about 41, 42, 43, 44, 45, 46, 47, 48, 49, or 50 weeks or more, and even up to an including a period of about 51, 52, 53, 54, 55, 56, 57, 58, 59, or 60, 70, 80, 90, 100, or even 200 or 300 weeks or more. As such, the inventors contemplate that particular therapeutic regimens involving one or more of the compositions disclosed herein will provide a biologically-effective amount of the IL-1Ra peptide, polypeptide, or protein, to the individual to which such compositions have been administered, for periods of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, and up to and including periods of therapy that persist in the treated individual for periods of at least about 1 year, at least about 2 years, at least about 3 years, at least about 4 years, year, at least about 6 years, at least about 7 years, at least about 8 years, at least about 9 years, or even at least about 10 or more years, up to and including the natural lifetime of the treated individual.

### 4.11 Exemplary Definitions

In accordance with the present invention, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the present invention, the preferred methods and compositions are described herein. For purposes of the present invention, the following terms are defined below:

A, an: In accordance with long standing patent law convention, the words “a” and “an” when used in this application, including the claims, denotes “one or more”.

Expression: The combination of intracellular processes, including transcription and translation undergone by a polynucleotide such as a structural gene to synthesize the encoded peptide or polypeptide.

Promoter: a term used to generally describe the region or regions of a nucleic acid sequence that regulates transcription.

Regulatory Element: a term used to generally describe the region or regions of a nucleic acid sequence that regulates transcription. Exemplary regulatory elements include, but are not limited to, enhancers, post-transcriptional elements, transcriptional control sequences, and such like.

Structural gene: A polynucleotide, such as a gene, that is expressed to produce an encoded peptide, polypeptide, protein, ribozyme, catalytic RNA molecule, siRNA, or antisense molecule.

Transformation: A process of introducing an exogenous polynucleotide sequence (e.g., a viral vector, a plasmid, or a recombinant DNA or RNA molecule) into a host cell or protoplast in which the exogenous polynucleotide is incorporated into at least a first chromosome or is capable of autonomous replication within the transformed host cell. Transfection, electroporation, and “naked” nucleic acid uptake all represent examples of techniques used to transform a host cell with one or more polynucleotides.

Transformed cell: A host cell whose nucleic acid complement has been altered by the introduction of one or more exogenous polynucleotides into that cell.

Transgenic cell: Any cell derived or regenerated from a transformed cell or derived from a transgenic cell, or from the progeny or offspring of any generation of such a transformed host cell.

Vector: A nucleic acid molecule (typically comprised of DNA) capable of replication in a host cell and/or to which another nucleic acid segment can be operatively linked so as to bring about replication of the attached segment. A plasmid, cosmid, or a virus is an exemplary vector.

The terms “substantially corresponds to”, “substantially homologous”, or “substantial identity” as used herein denotes a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides. Desirably, which highly homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by the hand of man in a laboratory is naturally-occurring. As used herein, laboratory strains of rodents that may have been selectively bred according to classical genetics are considered naturally occurring animals.

As used herein, a “heterologous” is defined in relation to a predetermined referenced gene sequence. For example, with respect to a structural gene sequence, a heterologous promoter is defined as a promoter which does not naturally occur adjacent to the referenced structural gene, but which is positioned by laboratory manipulation. Likewise, a heterologous gene or nucleic acid segment is defined as a gene or segment that does not naturally occur adjacent to the referenced promoter and/or enhancer elements.

“Transcriptional regulatory element” refers to a polynucleotide sequence that activates transcription alone or in combination with one or more other nucleic acid sequences. A transcriptional regulatory element can, for example, comprise one or more promoters, one or more response elements, one or more negative regulatory elements, and/or one or more enhancers.

As used herein, a “transcription factor recognition site” and a “transcription factor binding site” refer to a polynucleotide sequence(s) or sequence motif(s) which are identified as being sites for the sequence-specific interaction of one or more transcription factors, frequently taking the form of direct protein-DNA binding. Typically, transcription factor binding sites can be identified by DNA footprinting, gel mobility shift assays, and the like, and/or can be predicted on the basis of known consensus sequence motifs, or by other methods known to those of skill in the art.

As used herein, the term “operably linked” refers to a linkage of two or more polynucleotides or two or more nucleic acid sequences in a functional relationship. A nucleic acid is “operably linked” when it is placed into a functional relationship with another nucleic acid sequence. For instance, a promoter or enhancer is operably linked to a coding sequence if it affects the transcription of the coding sequence. “Operably linked” means that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

“Transcriptional unit” refers to a polynucleotide sequence that comprises at least a first structural gene operably linked to at least a first cis-acting promoter sequence and optionally linked operably to one or more other cis-acting nucleic acid sequences necessary for efficient transcription of the structural gene sequences, and at least a first distal regulatory element as may be required for the appropriate tissue-specific and developmental transcription of the structural gene sequence operably positioned under the control of the promoter and/or enhancer elements, as well as any additional cis sequences that are necessary for efficient transcription and translation (e.g., polyadenylation site(s), mRNA stability controlling sequence(s), etc.

The term “substantially complementary,” when used to define either amino acid or nucleic acid sequences, means that a particular subject sequence, for example, an oligonucleotide sequence, is substantially complementary to all or a portion of the selected sequence, and thus will specifically bind to a portion of an mRNA encoding the selected sequence. As such, typically the sequences will be highly complementary to the mRNA “target” sequence, and will have no more than about 1, 2, 3, 4, 5, 6, 7, 8, 9, or 10 base mismatches throughout the complementary portion of the sequence. In many instances, it may be desirable for the sequences to be exact matches, i.e. be completely complementary to the sequence to which the oligonucleotide specifically binds, and therefore have zero mismatches along the complementary stretch. As such, highly complementary sequences will typically bind quite specifically to the target sequence region of the mRNA and will therefore be highly efficient in reducing, and/or even inhibiting the translation of the target mRNA sequence into polypeptide product.

Substantially complementary oligonucleotide sequences will be greater than about 80 percent complementary (or ‘% exact-match’) to the corresponding mRNA target sequence to which the oligonucleotide specifically binds, and will, more preferably be greater than about 85 percent complementary to the corresponding mRNA target sequence to which the oligonucleotide specifically binds. In certain aspects, as described above, it will be desirable to have even more substantially complementary oligonucleotide sequences for use in the practice of the invention, and in such instances, the oligonucleotide sequences will be greater than about 90 percent complementary to the corresponding mRNA target sequence to which the oligonucleotide specifically binds, and may in certain embodiments be greater than about 95 percent complementary to the corresponding mRNA target sequence to which the oligonucleotide specifically binds, and even up to and including 96%, 97%, 98%, 99%, and even 100% exact match complementary to all or a portion of the target mRNA to which the designed oligonucleotide specifically binds.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) that are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

### 5. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 5.1 Example 1

The present example describes the result of studies using scAAV-based gene transfer as a means to treat various diseases and injuries of the joint. By delivering DNA encoding therapeutic agents to cells in and surrounding the joint tissues, the genetically modified cells are adapted to become factories for the sustained, localized production of the gene products. This strategy has been successful as a means to deliver therapeutic gene products to the joints of animals for the treatment of chronic joint diseases, such as arthritis.

The present example demonstrates that the use of AAV vectors that are self complementary (sc) (i.e., double stranded, containing both + and − DNA strands), overcome the limitation imposed by single-stranded vectors. scAAV vectors provide very high levels of gene expression with a rapid onset, both in vitro in fibroblastic cells cultured from the synovium and connective tissues comprising and surrounding the joint tissues (from humans, rats, rabbits and horses) and in vivo in the joints of experimental animals (rats, rabbits and horses).

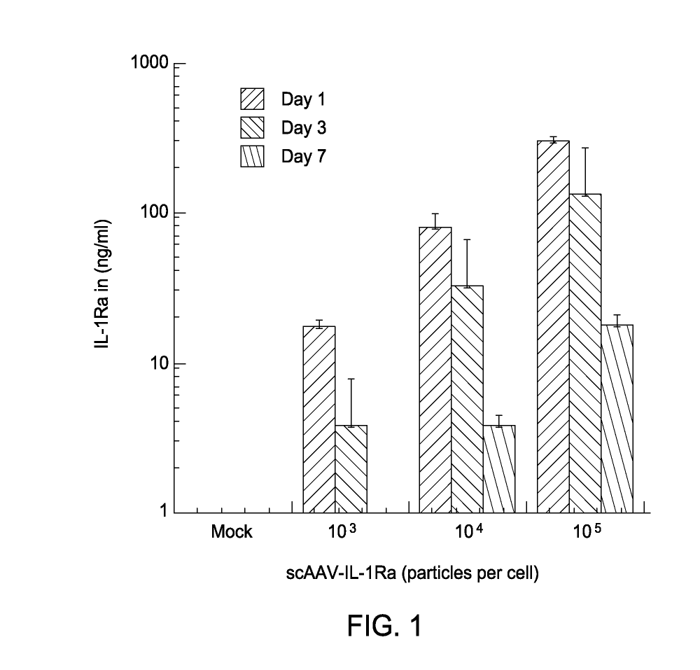

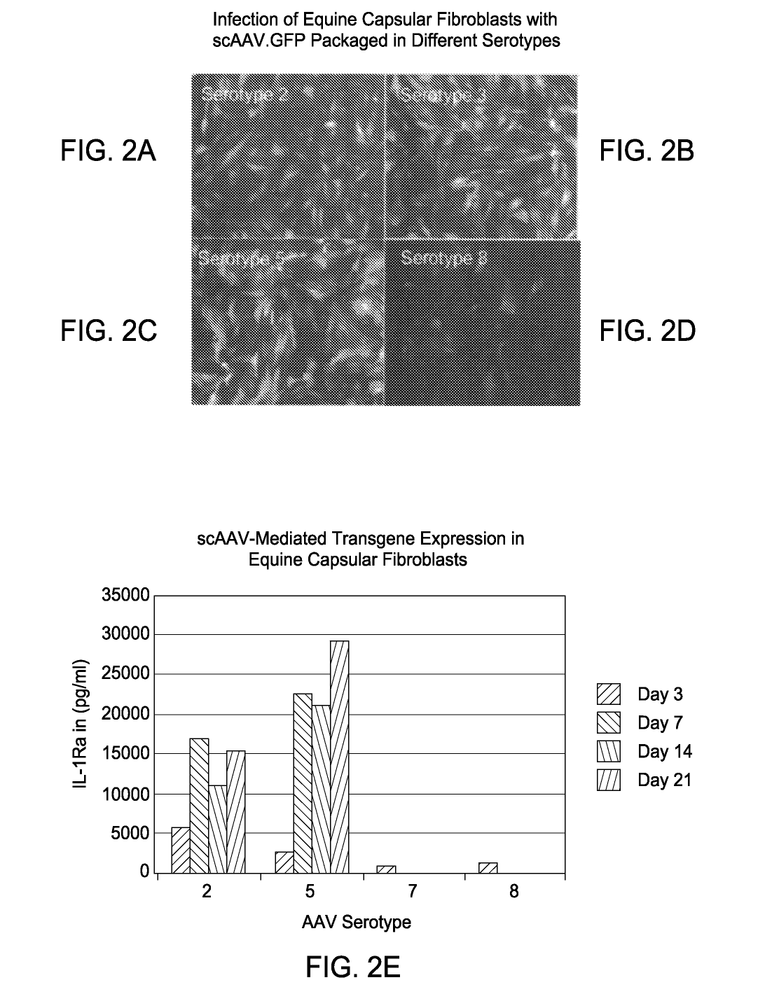

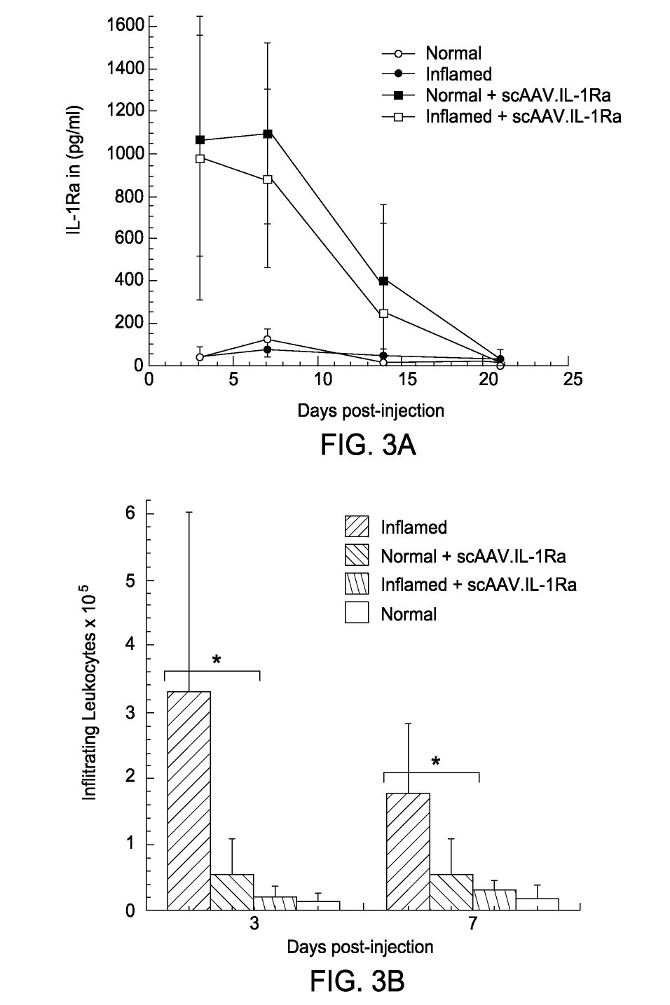

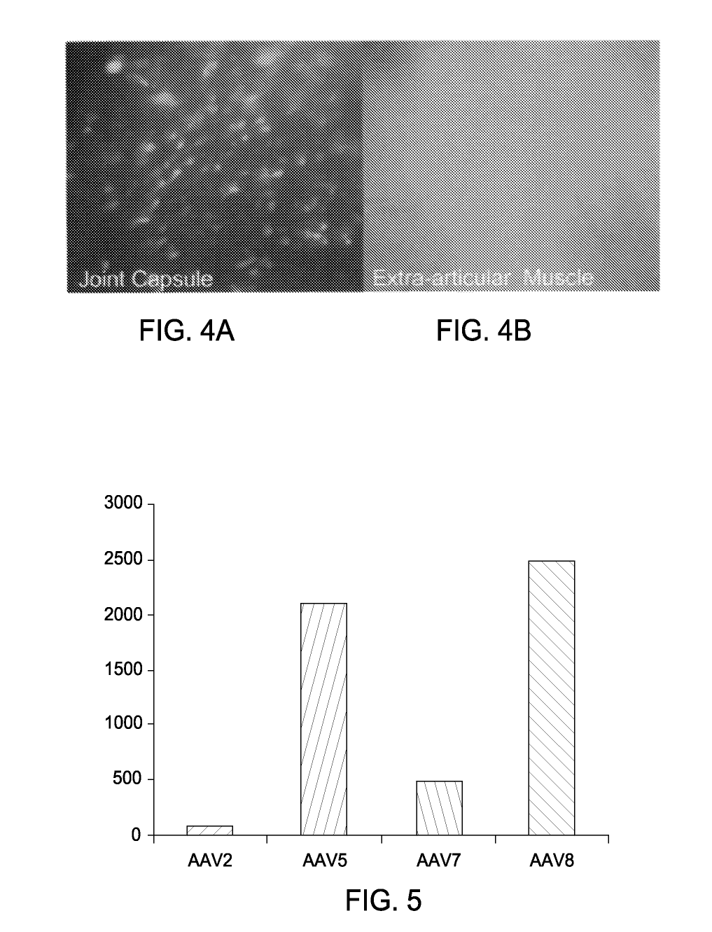

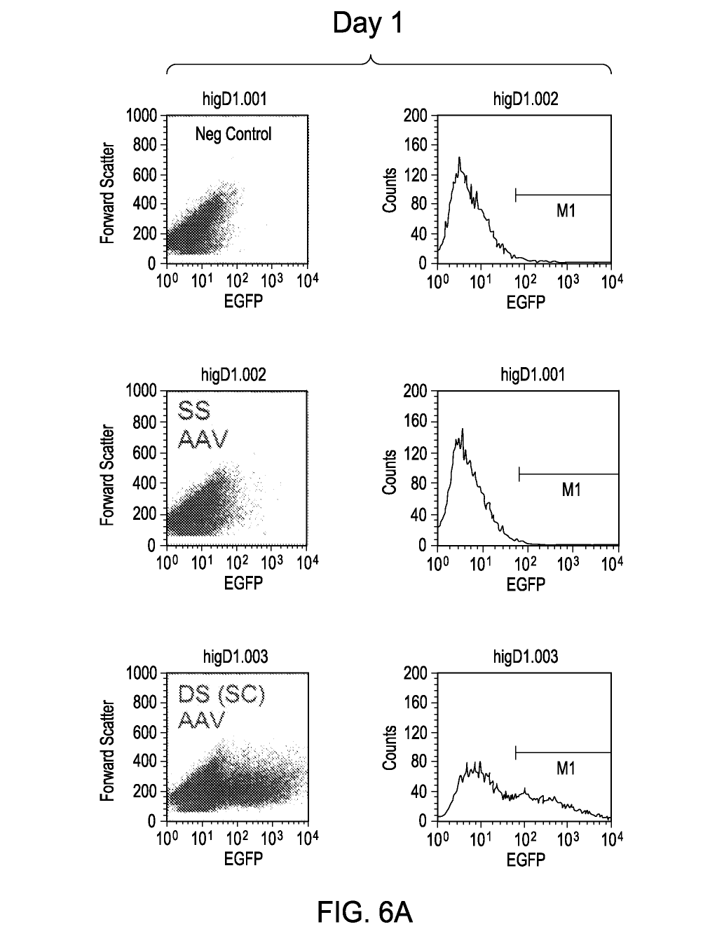

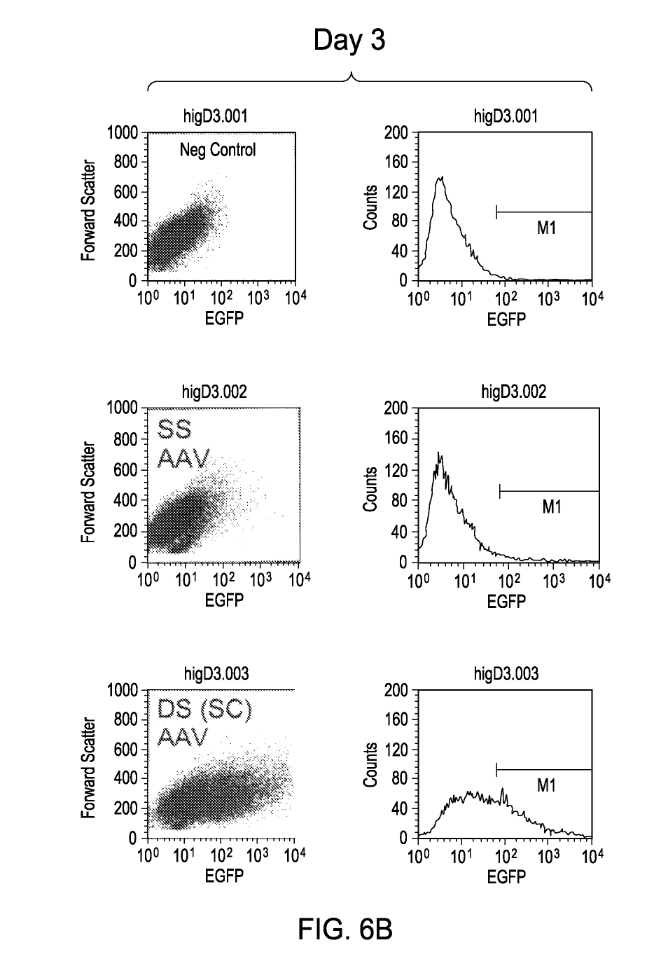

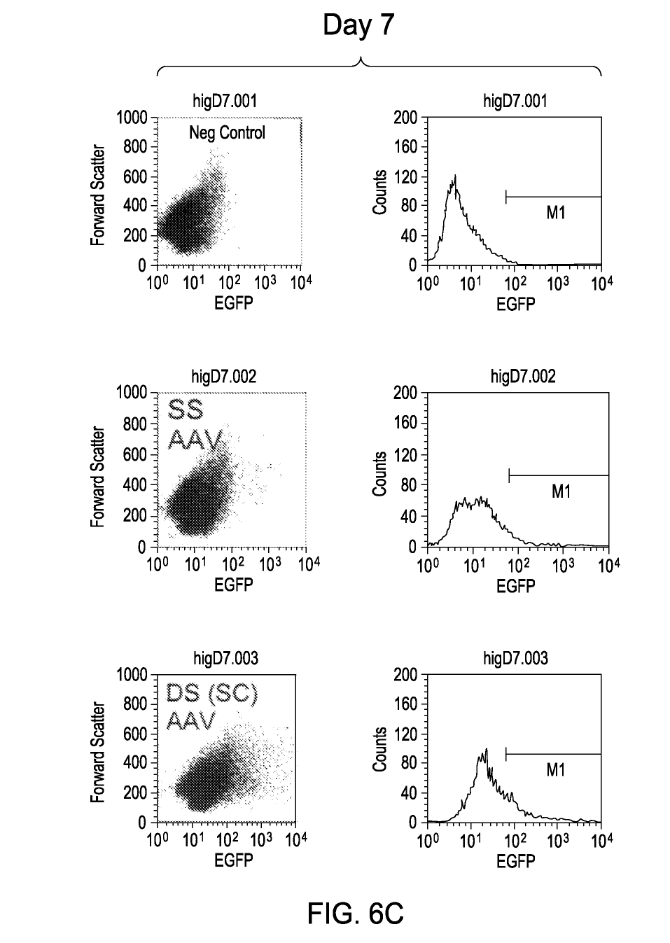

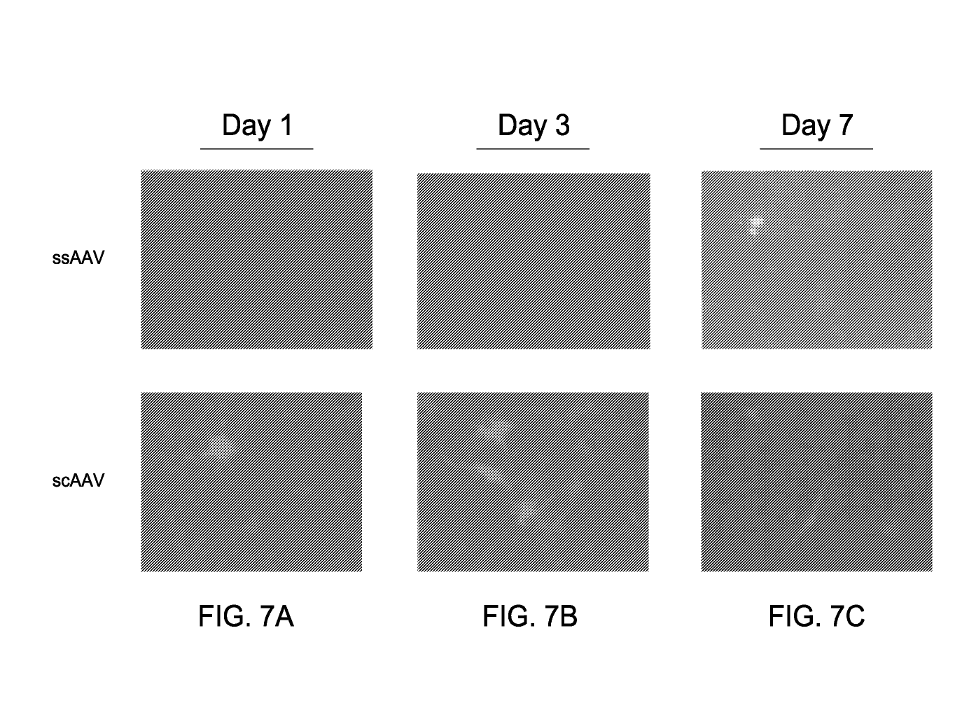

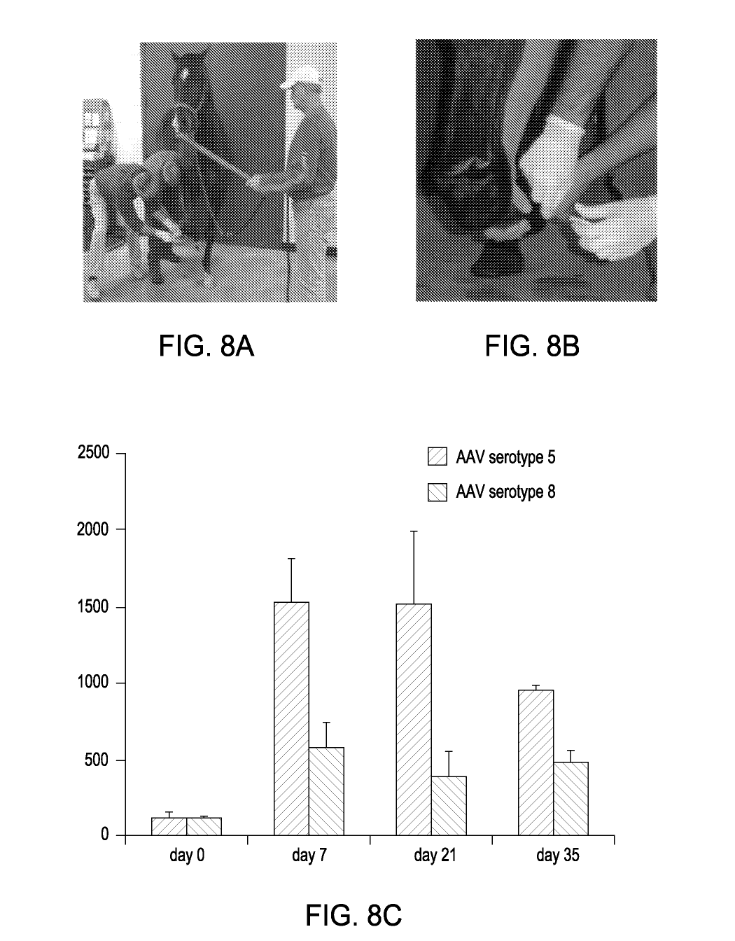

scAAV vectors can be produced either by generation of vector plasmids that are approximately half-genome sized combined with selective purification of the infectious double stranded form, or through the use of near half-genome sized vector plasmids with a mutation in one of the terminal resolution sequences of the AAV virus that provides for synthesis of double stranded virus. Both strategies generate + and − strand viral genomes that are covalently linked at one terminal repeat. Because the vector genomes are half wild type (˜2.2 kb) the resulting 2× viral construct (˜4.6) is able to be packaged into the normal AAV capsid. Both approaches to viral propagation provide a virus that enables a high level of functional transduction of capsular fibroblasts in culture. Examples are shown in FIG. 1 and FIG. 2 using human and equine cells, respectively. High functional levels of transgenic expression are achieved in vivo following intra-articular injection (FIG. 3A, FIG. 3B, FIG. 4A, and FIG. 4B), sufficient to mediate a beneficial therapeutic response intra-articularly (FIG. 3A and FIG. 3B).

### 5.2 Example 2

In studies using scAAV vectors, it was shown that following intra-articular injection, the AAV particles traverse the joint capsule to enable efficient transduction (genetic modification) of cells within the fibrous tissues of the joint capsule and in tissues that immediately surround the joint capsule. Gene transfer occurs primarily in cells of the periarticular muscle and connective tissues (FIG. 4A and FIG. 4B). These genetically modified cells then produce therapeutic transgene products proteins that through either diffusion or active transport mechanisms enter the joint space where the released gene products mediate a beneficial or therapeutic response. Because the large majority of gene transfer occurs in cells that are not part of the synovial lining, the genetically modified cells are not subject to the physical forces found intra-articularly. The cells are not rapidly lost and thus, are capable of stable expression of therapeutic transgene. This makes local, intra-articular gene delivery a viable and realistic approach for treating chronic joint conditions, such as arthritis which will require sustained, persistent expression of a therapeutic agent. Because the genetically modified cells are extra-articular, a significant portion of the transgene products that are secreted are also able to enter the circulation. Although the majority of transgenic expression localized within the joint region, this enables additional delivery of therapeutic transgene products to other sites in the body and in non-injected joints.

### 5.3 Example 3

Determining the levels and persistence of intra-articular expression enabled by the scAAV vector following delivery of homologous transgenes to the joints of experimental animals:

Several factors can contribute to limiting the expression of a transgene over time. Among the primary players are 1) immune elimination of transduced cells that express foreign (non-self) gene products. These can be in the form of cross species transgenes or native viral proteins from the vector. Since AAV vectors do not contain coding sequences for viral proteins, half of this concern is eliminated. Additional reasons are 2) loss of non-integrated viral genomes due to cell division and 3) loss of genetically modified cells due to natural turn over.

To determine the persistence of transgenic expression two studies have been developed. In the first, the rat homologue of the soluble tumor necrosis factor alpha receptor (ratTNFr) cDNA has been inserted into an scAAV plasmid vector. The scAAV-ratTNFr was then packaged into two capsid serotypes, 5 and 8. Approximately 5×10¹⁰ viral genomes were injected into the joints of normal Wistar rats. The rats are then sacrificed periodically and the expression of the transgene is measured following incubation of the dissected joint tissues overnight in culture media. Production of the ratTNFr protein in the conditioned media is then measured by ELISA. Short term data have shown increased expression of the ratTNFr protein in the joints receiving either the type 5 or the type 8 virus. Longer term data is collected over a period of 6 months to demonstrate persistence of the transgene expression in these animals.

The second study examines transgenic persistence in the joints of horses. Using the pHpa-trs-SK, scAAV plasmid as a delivery vehicle, the equine cDNAs for interleukin-1 receptor antagonist (IL-1Ra) and for insulin-like growth factor-1 (IGF-1) have been packaged into serotype 5 vectors. The equine IL-1Ra gene is expressed from the virus and its bioactivity is measurable. To follow persistence over time, the equine IGF-1 gene is being studied, which is only 2 amino acids different than the human form, and for which there exists a commercially available ELISA. Studies using scAAV-eqIGF-1 virus have been developed that are useful in determining long-term persistence of the therapeutic in the equine model.

### 5.4 Example 4

Self-complementary AAV (scAAV) provides improved transduction of rabbit capsular fibroblasts. Fibroblastic cells from the joints of rabbits were cultured in multiwell plates and infected with ˜1000 viral genomes per cell of either single stranded AAV containing the gene for GFP or scAAV packaged in serotype 2. At periodic intervals post infection individual cultures were analyzed for numbers of cells that expressed the GFP transgene and the levels of fluorescence. As shown in FIG. 6, the scAAV-GVP enabled significantly greater transduction of the rabbit cells. Expression was noted to diminish somewhat by day 7. This is due to the loss of episomal genomes from the rapidly dividing cells in culture.

### 5.5 Example 5

scAAV has also been demonstrated to provide improved transduction of rabbit capsular fibroblasts. Fibroblastic cells from the joints of rabbits were cultured in multiwell plates and infected with ˜1000 viral genomes per cell of either single stranded AAV (ssAAV) containing the gene for GFP or scAAV packaged in serotype 2. At periodic intervals post infection individual cultures were analyzed using fluorescence microscopy for numbers of cells that expressed the GFP transgene and the levels of fluorescence. As seen in FIG. 7, the single stranded vector provides very limited modification of the fibroblastic cells while the scAAV enables rapid onset of expression. Transgenic expression was observed to diminish over time due to the loss of episomal genomes from the rapidly dividing cells.

### 5.6 Example 6

scAAV-mediated gene transfer into equine joints has been shown to enable high-level expression of therapeutic transgene products. In an exemplary study, scAAV containing the cDNA for human IL-1Ra were packaged into AAV serotypes 5 or 8. As shown in FIG. 8C, scAAV packaged in both serotypes 5 and 8 enabled high levels of transgenic expression intra-articularly that persisted for at least one month. These experiments demonstrate that scAAV enables what are known to be functional levels of expression of therapeutic transgenes in large mammalian joints.

Recent studies are also aimed at determining the levels and persistence of expression attainable using homologous transgenes (i.e., same species). For these studies, the equine homologs of IL-1Ra and insulin-like growth factor-1 (IGF-1) have been inserted into the scAAV vector and have been used to generate recombinant virus. scAAV-eq-IGF-1 has been similarly injected into the joints of horses.

### 5.7 Example 7

### Mammalian Il-1RA Polypeptides and Nucleic Acid Segments Encoding Same

The inventors contemplate the use of one or more of the mammalian IL-1Ra polypeptide or polynucleotide sequences illustrated below (and disclosed herein in SEQ ID NO:1 through SEQ ID NO:21) in the preparation of scAAV vector-based constructs in the practice of the present invention. In illustrative embodiments, the inventors contemplate the use of mammalian IL-1Ra polypeptide-encoding polynucleotides, and in particular, the use of human IL-1Ra polypeptide-encoding polynucleotides (e.g., SEQ ID NO: 1) in creation of scAAV vector constructs that express a biologically-active IL-1Ra polypeptide (e.g, SEQ ID NO:2) for use in the preparation of medicaments, and in prophylactic and/or therapeutic regimens involving administration of such vector compositions for one or more articular disease(s) and/or dysfunction(s).

Homo sapiens (human) IL-1Ra Polynucleotide
Sequence (Gen Bank BC009745) (SEQ ID NO: 1):
ATGGCTTTAGAGACGATCTGCCGACCCTCTGGGAGAAAATCCAGCAAGAT
GCAAGCCTTCAGAATCTGGGATGTTAACCAGAAGACCTTCTATCTGAGGA
ACAACCAACTAGTTGCCGGATACTTGCAAGGACCAAATGTCAATTTAGAA
GAAAAGATAGATGTGGTACCCATTGAGCCTCATGCTCTGTTCTTGGGAAT
CCATGGAGGGAAGATGTGCCTGTCCTGTGTCAAGTCTGGTGATGAGACCA
GACTCCAGCTGGAGGCAGTTAACATCACTGACCTGAGCGAGAACAGAAAG
CAGGACAAGCGCTTCGCCTTCATCCGCTCAGACAGTGGCCCCACCACCAG
TTTTGAGTCTGCCGCCTGCCCCGGTTGGTTCCTCTGCACAGCGATGGAAG
CTGACCAGCCCGTCAGCCTCACCAATATGCCTGACGAAGGCGTCATGGTC
ACCAAATTCTACTTCCAGGAGGACGAGTAGTACTGCCCAGGCCTGCCTGT
TCCCATTCTTGCATGGCAA
Homo sapiens (human) IL-1Ra Polypeptide Sequence
(Gen Bank AAH09745.1) (SEQ ID NO:2):
MALETICRPSGRKSSKMQAFRIWDVNQKTFYLRNNQLVAGYLQGPNVNLE
EKIDVVPIEPHALFLGIHGGKMCLSCVKSGDETRLQLEAVNITDLSENRK
QDKRFAFIRSDSGPTTSFESAACPGWFLCTAMEADQPVSLTNMPDEGVMV
TKFYFQEDE
Homo sapiens (human) IL-1Ra Type 2 Polynucleotide
Sequence (Gen Bank NM_173343) (SEQ ID NO:3):
ATGTTGCGCTTGTACGTGTTGGTAATGGGAGTTTCTGCCTTCACCCTTCA
GCCTGCGGCACACACAGGGGCTGCCAGAAGCTGCCGGTTTCGTGGGAGGC
ATTACAAGCGGGAGTTCAGGCTGGAAGGGGAGCCTGTAGCCCTGAGGTGC
CCCCAGGTGCCCTACTGGTTGTGGGCCTCTGTCAGCCCCCGCATCAACCT
GACATGGCATAAAAATGACTCTGCTAGGACGGTCCCAGGAGAAGAAGAGA
CACGGATGTGGGCCCAGGACGGTGCTCTGTGGCTTCTGCCAGCCTTGCAG
GAGGACTCTGGCACCTACGTCTGCACTACTAGAAATGCTTCTTACTGTGA
CAAAATGTCCATTGAGCTCAGAGTTTTTGAGAATACAGATGCTTTCCTGC
CGTTCATCTCATACCCGCAAATTTTAACCTTGTCAACCTCTGGGGTATTA
GTATGCCCTGACCTGAGTGAATTCACCCGTGACAAAACTGACGTGAAGAT
TCAATGGTACAAGGATTCTCTTCTTTTGGATAAAGACAATGAGAAATTTC
TAAGTGTGAGGGGGACCACTCACTTACTCGTACACGATGTGGCCCTGGAA
GATGCTGGCTATTACCGCTGTGTCCTGACATTTGCCCATGAAGGCCAGCA
ATACAACATCACTAGGAGTATTGAGCTACGCATCAAGAAAAAAAAAGAAG
AGACCATTCCTGTGATCATTTCCCCCCTCAAGACCATATCAGCTTCTCTG
GGGTCAAGACTGACAATCCCGTGTAAGGTGTTTCTGGGAACCGGCACACC
CTTAACCACCATGCTGTGGTGGACGGCCAATGACACCCACATAGAGAGCG
CCTACCCGGGAGGCCGCGTGACCGAGGGGCCACGCCAGGAATATTCAGAA
AATAATGAGAACTACATTGAAGTGCCATTGATTTTTGATCCTGTCACAAG
AGAGGATTTGCACATGGATTTTAAATGTGTTGTCCATAATACCCTGAGTT
TTCAGACACTACGCACCACAGTCAAGGAAGCCTCCTCCACGTTCTCCTGG
GGCATTGTGCTGGCCCCACTTTCACTGGCCTTCTTGGTTTTGGGGGGAAT
ATGGATGCACAGACGGTGCAAACACAGAACTGGAAAAGCAGATGGTCTGA
CTGTGCTATGGCCTCATCATCAAGACTTTCAATCCTATCCCAAGTGA
Homo sapiens (human) IL-1Ra Type 2 Polypeptide
Sequence (Gen Bank P_775465.1) (SEQ ID NO:4):
MLRLYVLVMGVSAFTLQPAAHTGAARSCRFRGRHYKREFRLEGEPVALRC
PQVPYWLWASVSPRINLTWHKNDSARTVPGEEETRMWAQDGALWLLPALQ
EDSGTYVCTTRNASYCDKMSIELRVFENTDAFLPFISYPQILTLSTSGVL
VCPDLSEFTRDKTDVKIQWYKDSLLLDKDNEKFLSVRGTTHLLVHDVALE
DAGYYRCVLTFAHEGQQYNITRSIELRIKKKKEETIPVIISPLKTISASL
GSRLTIPCKVFLGTGTPLTTMLWWTANDTHIESAYPGGRVTEGPRQEYSE
NNENYIEVPLIFDPVTREDLHMDFKCVVHNTLSFQTLRTTVKEASSTFSW
GIVLAPLSLAFLVLGGIWMHRRCKHRTGKADGLTVLWPHHQDFQSYPK
Rattus norvegicus (Norway rat) IL-1Ra
Polynucleotide Sequence (Gen Bank NM_022194)
(SEQ ID NO:5):
GCAAGCCTTCAGAATCTGGGATACTAACCAGAAGACCTTCTACCTGAGGA
ACAACCAGCTCATTGCTGGGTACTTACAAGGACCAAATACCAAACTAGAA
GAAAAGATAGACATGGTGCCTATTGACTTTCGGAATGTGTTCTTGGGCAT
CCACGGGGGCAAGCTGTGCCTGTCTTGTGTCAAGTCTGGAGATGACACCA
AGCTCCAGCTGGAGGAGGTTAACATCACTGATCTGAACAAGAACAAAGAA
GAAGACAAGCGCTTTACCTTCATCCGCTCCGAG
Rattus norvegicus (Norway rat) IL-1Ra Polypeptide
Sequence (GenBank NP_071530.1) (SEQ ID NO:6):
MEICRGPYSHLISLLLILLFRSESAGHPAGKRPCKMQAFRIWDTNQKTFY
LRNNQLIAGYLQGPNTKLEEKIDMVPIDFRNVFLGIHGGKLCLSCVKSGD
DTKLQLEEVNITDLNKNKEEDKRFTFIRSETGPTTSFESLACPGWFLCTT
LEADHPVSLTNTPKEPCTVTKFYFQEDQ
Canis lupus familiaris (Dog) IL-1Ra Polynucleotide
Sequence (GenBank NM_001003096) (SEQ ID NO:7):
ACCGAATGGAAACCTGCAGGTGTCCTCTCAGCTACCTAATCTCTTTCCTC
CTTTTCCTGTCCCATTCAGAGACAGCCTGCCGTCCCTTGGGGAAGAGACC
TTGCAGGATGCAAGCCTTCAGAATCTGGGATGTTAACCAGAAGACCTTCT
ACCTGAGGAATAACCAACTAGTCGCTGGATACTTGCAAGGATCAAATACT
AAATTAGAAGAGAAGTTAGATGTGGTGCCCGTCGAGCCTCATGCCGTGTT
CTTGGGGATCCATGGGGGGAAGCTGTGCCTGGCCTGTGTCAAGTCTGGAG
ATGAGACCAGGCTCCAGCTGGAGGCCGTTAACATCACTGACCTGAGTAAG
AACAAGGATCAAGACAAGCGCTTTACCTTCATCCTCTCAGACAGTGGCCC
TACCACCAGCTTTGAGTCTGCTGCCTGCCCTGGCTGGTTCCTCTGCACAG
CACTGGAGGCCGACCGGCCTGTCAGCCTCACCAACAGACCAGAAGAGGCC
ATGATGGTCACTAAGTTCTACTTCCAGAAGGAATAA
Canis lupus familiaris (Dog) IL-1Ra Polypeptide
Sequence (GenBank NP_001003096.1) (SEQ ID NO:8):
METCRCPLSYLISFLLFLSHSETACRPLGKRPCRMQAFRIWDVNQKTFYL
RNNQLVAGYLQGSNTKLEEKLDVVPVEPHAVFLGIHGGKLCLACVKSGDE
TRLQLEAVNITDLSKNKDQDKRFTFILSDSGPTTSFESAACPGWFLCTAL
EADRPVSLTNRPEEANNVTKFYFQKE
Oryctolagus cuniculus (Rabbit) IL-1Ra
Polynucleotide Sequence (GenBank NM_001082770)
(SEQ ID NO:9):
ATGAGACCCTCCAGGAGCACCCGCAGGCACCTAATCTCCCTCCTCCTCTT
CCTGTTCCATTCAGAGACAGCCTGCCGCCCTTCTGGGAAAAGACCTTGCA
GGATGCAGGCCTTCAGAATCTGGGATGTTAACCAGAAGACCTTCTACTTG
AGAAACAACCAACTAGTCGCTGGTTACTTGCAAGGCCCAAATGCCAAATT
AGAAGAAAGGATAGATGTGGTGCCCCTTGAGCCTCAGCTCCTGTTCCTGG
GCATCCAGAGGGGGAAGTTGTGCCTGTCTTGTGTGAAGTCTGGGGATAAG
ATGAAGCTCCATTTGGAGGCCGTTAACATCACTGACCTGGGCAAGAACAA
GGAGCAGGACAAGCGCTTCACCTTCATCCGCTCCAATAGCGGCCCTACCA
CCACCTTCGAGTCTGCCTCCTGCCCGGGCTGGTTTCTCTGCACGGCCCTG
GAGGCTGACCAGCCCGTCAGCCTCACCAACACGCCCGACGACTCCATCGT
GGTCACCAAGTTCTACTTCCAGGAGGACCAGTAG
Oryctolagus cuniculus (Rabbit) IL-1Ra Polypeptide
Sequence (GenBank P_001076239.1) (SEQ ID NO:10):
MRPSRSTRRHLISLLLFLFHSETACRPSGKRPCRMQAFRIWDVNQKTFYL
RNNQLVAGYLQGPNAKLEERIDVVPLEPQLLFLGIQRGKLCLSCVKSGDK
MKLHLEAVNITDLGKNKEQDKRFTFIRSNSGPTTTFESASCPGWFLCTAL
EADQPVSLTNTPDDSIVVTKFYFQEDQ
Equus caballus (Horse) IL-1Ra Polynucleotide
Sequence (GenBank M_001082525) (SEQ ID NO:11):
ATGGAAATCCGCAGGCGTTCTGTCAGACACCTAATCTCTCTCCTCCTTTT
CTTGCTCTACTCAGAGACAGCCTGCCACCCTTTGGGGAAGAGACCCTGCA
AGATGCAAGCCTTCAGAATCTGGGATGTTAACCAGAAGACCTTCTACATG
AGGAATAACCAACTAGTTGCTGGATACTTGCAAGAATCAAATACTAAATT
ACAAGAGAAGATAGATGTGGTGCCCATTGAGCCTGATGCTCTATTCCTGG
GACTCCATGGGAGGAAGCTGTGCCTGGCCTGTGTCAAGTCTGGTGATGAG
ATTAGGTTCCAATTGGAGGCAGTTAACATCACTGACCTGAGCAAGAACAA
GGAGGAGAACAAGCGCTTCACCTTCATCCGCTCAAACAGTGGCCCCACCA
CCAGCTTCGAGTCTGCCGCCTGCCCTGGCTGGTTCCTCTGCACGGCGCAG
GAGGCAGACCGGCCCGTCAGCCTCACCAACAAGCCCAAAGAGTCCTTCAT
GGTCACCAAGTTCTACCTCCAGGAGGACCAGTAG
Equus caballus (Horse) IL1Ra Polypeptide Sequence
(GenBank NP_001075994.1) (SEQ ID NO:12).
MEIRRRSVRHLISLLLFLLYSETACHPLGKRPCKMQAFRIWDVNQKTFYM
RNNQLVAGYLQESNTKLQEKIDVVPIEPDALFLGLHGRKLCLACVKSGDE
IRFQLEAVNITDLSKNKEENKRFTFIRSNSGPTTSFESAACPGWFLCTAQ
EADRPVSLTNKPKESFMVTKFYLQEDQ
Sus scrofa (Pig) IL-1Ra Polynucleotide Sequence
(GenBank NM_214262) (SEQ ID NO:13):
ATGGAAGTTAGCAGGTATCTGTGCAGTTACCTAATCTCTTTCCTCCTTTT
CCTGTTCCACTCAGAGACTGCCTGCCACCCCTTGGGAAAGAGACCTTGCA
GGATGCAAGCCTTCAGAATCTGGGATGTCAACCAGAAGACTTTCTACCTG
AGGAATAACCAATTAGTTGCTGGATACTTGCAAGGACCAAATACTAAACT
GGAAGAGAAGATAGATGTGGTGCCTGTTGAGCCTCATTTTGTGTTCCTGG
GGATCCATGGAGGGAAGCTGTGCCTGTCCTGTGTCAAGTCTGGTGATGAG
ATGAAGCTCCAGTTGGACGCAGTTAACATCACAGACCTGAGAAAGAACAG
CGAGCAGGACAAGCGCTTCACCTTCATCCGCTCCGACAGTGGCCCCACCA
CCAGCTTTGAGTCAGCCGCCTGTCCTGGCTGGTTCCTCTGCACTGCACTA
GAGGCAGACCAGCCTGTTGGCCTCACCAACACGCCCAAAGCAGCCGTCAA
GGTCACCAAGTTCTACTTCCAGCAGGACCAGTAA
Sus scrofa (Pig) IL-1Ra Polypeptide Sequence
(GenBank accession NM_214262.1) (SEQ ID NO:14):
MEVSRYLCSYLISFLLFLFHSETACHPLGKRPCRMQAFRIWDVNQKTFYL
RNNQLVAGYLQGPNTKLEEKIDVVPVEPHFVFLGIHGGKLCLSCVKSGDE
MKLQLDAVNITDLRKNSEQDKRFTFIRSDSGPTTSFESAACPGWFLCTAL
EADQPVGLTNTPKAAVKVTKFYFQQDQ
Mus musculus (Mouse) IL-1Ra Polynucleotide
Sequence (GenBank BC042532) (SEQ ID NO:15):
ATGGCTTCAGAGGCAGCCTGCCGCCCTTCTGGGAAAAGACCCTGCAAGAT
GCAAGCCTTCAGAATCTGGGATACTAACCAGAAGACCTTTTACCTGAGAA
ACAACCAGCTCATTGCTGGGTACTTACAAGGACCAAATATCAAACTAGAA
GAAAAGTTAGACATGGTGCCTATTGACCTTCATAGTGTGTTCTTGGGCAT
CCACGGGGGCAGCTGTGCCTGTCTTGTGCCAAGTCTGGAGATGATATCAA
GCTCCAGCTGGAGGAAGTTAACATCACTGATCTGAGCAAGAACAAAGAAG
AAGACAAGCGCTTTACCTTCATCCGCTCTGAGAAAGGCCCCACCACCAGC
TTTGAGTCAGCTGCCTGTCCAGGATGGTTCCTCTGCACAACACTAGAGGC
TGACCGTCCTGTGAGCCTCACCAACACACCGGAAGAGCCCCTTATAGTCA
CGAAGTTCTACTTCCAGGAAGACCAATAG
Mus musculus (Mouse) IL-1Ra Polypeptide Sequence
(GenBank AAH42532.1) (SEQ ID NO:16):
MASEAACRPSGKRPCKMQAFRIWDTNQKTFYLRNNQLIAGYLQGPNIKLE
EKLDMVPIDLHSVFLGIHGGKLCLSCAKSGDDIKLQLEEVNITDLSKNKE
EDKRFTFIRSEKGPTTSFESAACPGWFLCTTLEADRPVSLTNTPEEPLIV
TKFYFQEDQ
Bos taurus (Cow) IL-1Ra Polynucleotide Sequence
(GenBank NM_174357) (SEQ ID NO:17):
ATGGACATCTACATCCACGGTTACCTAATCTGTCTTCTCCTCTTCCTGTT
CCGTTCAGAAACAGCCTGCCACCCCTTGGGAAAGAGACGCTGCGAGATGC
AAGCCTTCAGGATCTGGGATGTCAACCAGAAGATCTTCTACCTGAGGAAT
AACCAATTAGTTGCTGGATACTTGCAAGGACCAAATACTAAATTAGAGGA
GAAGATAGATGTGGTACCCATCGAACCCCATACTATGTTCCTGGGGATCC
ATGGGGGGAAGCTGTGCCTGGCCTGCGTAAAATCTGGAGATGAGATCAAG
CTCAAGTTAGAGGCCGTGAACATCACTGACTTGAACCAGAACAGGGAGCA
GGACAAGCGCTTTGCCTTCATCCGCTTCGACAACGGGCCCACCACCAGCT
TTGAGTCAGCTGCCTGCCCCGGCTGGTTCCTCTGCACATCACTGGAGGCC
GACCAGCCCGTGGGCCTCACCAATATGCCCACAGAGGCCCTCAAGGTCAC
CAAGTTCTACTTCCAGCAGGACTAG
Bos taurus (Cow) IL-1Ra Polypeptide Sequence
(GenBank NP_776782.1) (SEQ ID NO: 18):
MDIYIHGYLICLLLFLFRSETACHPLGKRRCEMQAFRIWDVNQKIFYLRN
NQLVAGYLQGPNTKLEEKIDVVPIEPHTMFLGIHGGKLCLACVKSGDEIK
LKLEAVNITDLNQNREQDKRFAFIRFDNGPTTSFESAACPGWFLCTSLEA
DQPVGLTNMPTEALKVTKFYFQQD
Macaca fascicularis (Macaque) IL -1Ra Polypeptide
Sequence (GenBank AA024703) (SEQ ID NO:19):
MEICRGLGSHLICLLLFLFHSETVGRPSGRKPSKMQAFRIWDVNQKTFYL
RNNQLVAGYLQGPNVNLEEKIDVVPIEPHALFLGIHGGKMCLSCVKSGDE
TRLQLEAVNITDLSKNRKQDKRFAFVRSDSGPTTSFESAACPGWFLCTAM
EADQPVSLTNMPDKGVMVTKFYFQEDE
Macaca mulatta (Rhesus monkey) IL-1Ra Polypeptide
Sequence (GenBank XP_001091717) (SEQ ID NO:20):
MALETVGRPSGRKPSKMQAFRIWDVNQKTFYLRNNQLVAGYLQGPNVNLE
EKIDVVPIEPHALFLGIHGGKMCLSCVKSGDETRLQLEAVNITDLSKNRK
QDKRFAFVRSDSGPTTSFESAACPGWFLCTAMEADQPVSLTNMPDKGVMV
TKFYFQEDE
Monodelphis domestica (gray short-tailed opossum)
IL-1Ra Polypeptide Sequence (GenBank XP_001376878)
(SEQ ID NO:21):
MDLSVCPRSYLITLFLFLFHSETESFPFVKMSSKRQAYRIWDIYQKTFYL
RNNQLVAGYLQGSNTNLEEKIDLVSIEPDSVLLGIHDGRLGLACVKTGGT
PKLQLEAVNITDLDKNQQESKRFTFIKSENDSICRFESAACPGWFLCTQT
ESDRPVGLSNTQANTITRFYFQRE

It will be apparent to those skilled in the art that various changes may be made in the invention without departing from the spirit and scope thereof, and therefore, the invention encompasses embodiments in addition to those specifically disclosed in the specification.

### 6. REFERENCES

The following references, to the extent that they provide exemplary procedural or other details supplementary to those set forth herein, are specifically incorporated herein by reference.
1. Evans, C H; Ghivizzani, S C; Kang, R; Muzzonigro, T; Wasko, M C; Herndon, J H; and Robbins, P D. “Gene therapy for rheumatic diseases,” Arthritis. Rheum., 42:1-16, 1999.
2. Ghivizzani, S C; Oligino, T J; Glorioso, J C; Robbins, P D; and Evans, C H. “Direct gene delivery strategies for the treatment of rheumatoid arthritis,” D rug Discov. Today, 6:259-267, 2001.
3. Arend, W P. “Physiology of cytokine pathways in rheumatoid arthritis,” Arthritis Rheum., 45:101-106, 2001.
4. Le, C H; Nicolson, A G; Morales, A; and Sewell, K L. “Suppression of collagen-induced arthritis through adenovirus-mediated transfer of a modified tumor necrosis factor alpha receptor gene,” Arthritis Rheum., 40:1662-1669, 1997.
5. Bessis, N; Chiocchia, G; Kollias, G; Minty, A; Fournier, C; Fradelizi, D; and Boissier, MC. “Modulation of proinflammatory cytokine production in tumour necrosis factor-alpha (TNF-α)-transgenic mice by treatment with cells engineered to secrete IL-4, IL-10 or IL-13,” Clin. Exp. Immunol., 111:391-396, 1998 [Erratum published Clin. Exp. Immunol., 113(1):145, 1998].
6. Boggs, S S; Patrene, K D; Mueller, G M; Evans, C H; Doughty, L A; and Robbins, P D. “Prolonged systemic expression of human IL-1 receptor antagonist (hIL-1RA) in mice reconstituted with hematopoietic cells transduced with a retrovirus carrying the HIL-1RA cDNA,” Gene Ther., 2:632-638, 1995.
7. Kim, S H; Kim, S; Evans, C H; Ghivizzani, S C; Oligino, T; and Robbins, P D. “Effective treatment of established murine collagen-induced arthritis by systemic administration of dendritic cells genetically modified to express IL-4,” J. Immunol., 166:3499-3505, 2001.
8. Evans, C H; Ghivizzani, S C; and Robbins, P D. “Blocking cytokines with genes,” J. Leukoc. Biol., 64:55-61, 1998.
9. Evans, C H; Bandara, G; Mueller, G; Robbins, P; Glorioso, J C; and Georgescu, H I. “Synovial cell transplants for gene transfer to joints,” Transplant. Proc., 24:2966.1992.
10. Edwards, J C W. “Fibroblastic synovial lining cells (synoviocytes). In: Henderson, B; Edwards, J C W; and Pettipher, E R (Eds). Mechanisms and Models in Rheumatoid Arthritis. Academic Press: San Diego, pp. 153-162, 1995.
11. Edwards, J C W; and Wilkinson, LS. “Immunohistochemistry of rheumatoid synovium. In: Henderson, B; Edwards, J C W, and Pettipher, E R; (Eds). Mechanisms and Models in Rheumatoid Arthritis. Academic Press: San Diego, pp. 133-150, 1995.
12. Revell, P A; and Mapp, P I. “Synovial lining cells. ultrastructural characterisation of macrophages (type A cells) in the synovial lining,” Rheumatol. Intl., 9:49-51, 1989.
13. Hung, G L; and Evans, C H. “Synovium. In: Fu, F H; Harner, C D; and Vince, K G. (Eds) Knee Surg, Williams & Wilkins: Baltimore, pp. 141-154, 1994.
14. Zimmermann, T; Kunisch, E; Pfeiffer, R; Hirth, A; Stahl, H D; Sack, U; Laube, A; Liesaus, E; Roth, A; Palombo-Kinne, E; Emmrich, F; and Kinne, R W. “Isolation and characterization of rheumatoid arthritis synovial fibroblasts from primary culture—primary culture cells markedly differ from fourth-passage cells,” Arthritis Res., 3:72-76, 2001.
15. Baddour, V T; Bradley, JD. “Clinical assessment and significance of inflammation in knee osteoarthritis,” Curr. Rheumatol. Rept, 1:59-63, 1999.
16. Verschure, P J; and Van Noorden, C J. “The effects of interleukin-1 on articular cartilage destruction as observed in arthritic diseases, and its therapeutic control, Clin. Exp. Rheumatol., 8:303-313, 1990.
17. Goldring, M B. “Anticytokine therapy for osteoarthritis,” Expert Opin. Biol. Ther., 1:817-829, 2001.
18. Hardingham, T E; Venn, G; and Bayliss, M T. “Chondrocyte responses in cartilage and in experimental osteoarthritis, Brit. J. Rheum., 30(Suppl 1):32-37, 1991.
19. van den Berg, W B. “Arguments for interleukin 1 as a target in chronic arthritis,” Ann. Rheum. Dis., 59(Suppl 1):i81-84, 2000.
20. Evans, C H; and Robbins, P D. “Progress toward the treatment of arthritis by gene therapy,” Ann. Med., 27:543-546, 1995.
21. Mohr, W; Beneke, G; and Mohing, W. “Proliferation of synovial lining cells and fibroblasts,” Ann. Rheum. Dis., 34:219-224, 1975.
22. Coulton, L A; Henderson, B; Bitensky, L; and Chayen, J. “DNA synthesis in human rheumatoid and nonrheumatoid synovial lining,” Ann. Rheum. Dis., 39:241-247, 1980.
23. Muller-Ladner, U; Roberts, C R; Franklin, B N; Gay, R E; Robbins, P D; Evans, C H; and Gay, S. “Human IL-1Ra gene transfer into human synovial fibroblasts is chondroprotective,” J. Immunol., 158:3492-3498, 1997.
24. Bandara, G; Mueller, G M; Galea-Lauri, J; Tindal, M H; Georgescu, H I; Suchanek, M K; Hung, G L; Glorioso, J C; Robbins, P D; and Evans, C H. “Intraarticular expression of biologically active interleukin 1-receptor-antagonist protein by ex vivo gene transfer,” Proc. Natl. Acad. Sci. USA, 90:10764-10768, 1993.
25. Hung, G L; Galea-Lauri, J; Mueller, G M; Georgescu, H I; Larkin, L A; Suchanek, M K; Tindal, M H; Robbins, P D; and Evans, C H. “Suppression of intra-articular responses to interleukin-1 by transfer of the interleukin-1 receptor antagonist gene to synovium,” Gene Ther., 1:64-69, 1994.
26. Otani, K; Nita, I; Macaulay, W; Georgescu, H I; Robbins, P D; and Evans, C H. “Suppression of antigen-induced arthritis in rabbits by ex vivo gene therapy,” J. Immunol., 156:3558-3562, 1996.
27. Makarov, S S; Olsen, J C; Johnston, W N; Anderle, S K; Brown, R R; Baldwin, A S, Jr.; Haskill, J S; and Schwab, J H. “Suppression of experimental arthritis by gene transfer of interleukin 1 receptor antagonist cDNA,” Proc. Natl. Acad. Sci. USA, 93:402-406, 1996.
28. Evans, C H; Robbins, P D; Ghivizzani, S C; Herndon, J H; Kang, R; Bahnson, A B; Barranger, J A; Elders, E M; Gay, S; Tomaino, M M; Wasko, M C; Watkins, S C; Whiteside, T L; Glorioso, J C; Lotze, M T; and Wright, T M. “Clinical trial to assess the safety, feasibility, and efficacy of transferring a potentially anti-arthritic cytokine gene to human joints with rheumatoid arthritis,” Hum. Gene Ther., 7:1261-1280, 1996.
29. Evans, C H; Robbins, P D; Ghivizzani, S C; Wasko, M C; Tomaino, M X; Kang, R; Muzzonigro, T A; Voigt, M; Elder, E M; Whiteside, T L; Watkins, S C; and Herndon, J H. “Gene transfer to human joints: progress towards a gene therapy of arthritis,” Proc. Natl. Acad. Sci. USA, 102(24):8698-703, 2005.
30. Veelken H, Jesuiter H, Mackensen A, Kulmburg P, Schultze J, Rosenthal F, Mertelsmann R, Lindemann, A. “Primary fibroblasts from human adults as target cells for ex vivo transfection and gene therapy,” Hum. Gene Ther., 5:1203-1210, 1994.
31. Kang W K, Park C, Yoon H L, Kim W S, Yoon S S, Lee M H, Park K, Kim K, Jeong H S, Kim J A, Nam S J, Yang J H, Son Y I, Baek C H, Han J, Ree H J, Lee E S, Kim S H, Kim D W, Ahn Y C, Huh S J, Choe Y H, Lee J H, Park M H, Kong G S et al., “Interleukin 12 gene therapy of cancer by peritumoral injection of transduced autologous fibroblasts: outcome of a phase I study,” Hum. Gene Ther., 12:671-684, 2001.
32. Caplan A I. “Mesenchymal stem cells and gene therapy,” Clin. Orthop., S67-70, 2000.
33. Mosca J D, Hendricks J K, Buyaner D, Davis-Sproul J, Chuang L C, Majumdar M K, Chopra R, Barry F, Murphy M, Thiede M A, Junker U, Rigg R J, Forestell S P, Bohnlein E, Storb R, Sandmaier B M. “Mesenchymal stem cells as vehicles for gene delivery,” Clin. Orthop., S71-90, 2000.
34. Ghivizzani S C, Lechman E R, Kang R, Tio C, Kolls J, Evans C H, Robbins P D. “Direct adenovirus-mediated gene transfer of interleukin 1 and tumor necrosis factor alpha soluble receptors to rabbit knees with experimental arthritis has local and distal anti-arthritic effects,” Proc. Natl. Acad. Sci. USA, 95:4613-4618, 1998.
35. Schiedner G, Kochanek S, Castro M G, Lowenstein P R. “Development of high-capacity adenoviral vectors for gene therapy,” Proc. Natl. Acad. Sci. USA, 97:7482-7487, 2000.
36. Jiang Z L, Reay D, Kreppel F, Gambotto A, Feingold E, Kochanek S, McCarthy S A, Clemens P R, Thomas C E. “Local high-capacity adenovirus-mediated mCTLA4Ig and mCD40Ig expression prolongs recombinant gene expression in skeletal muscle,” Mol. Ther., 3(6):892-900, 2001.
Thomas, C E; Schiedner, G; Kochanek, S; Castro, M G; and Lowenstein, P R. “Preexisting antiadenoviral immunity is not a barrier to efficient and stable transduction of the brain, mediated by novel high-capacity adenovirus vectors,” Hum. Gene Ther., 12(7): 839-846.
37. Floyd, S S; Clemens P R; Ontell, M R; Kochanek, S; Day, CS; Yang, J; Hauschka, S D; Balkir, L; Morgan, J; Moreland, M S; Feero, G W; Epperly, M; and Huard, J. “Ex vivo gene transfer using adenovirus-mediated full-length dystrophin delivery to dystrophic muscles,” Gene Ther., 5:19-30, 1998.
38. Vigna, E; and Naldini, L. “Lentiviral vectors: excellent tools for experimental gene transfer and promising candidates for gene therapy,” J Gene Med, 2:308-316, 2000.
39. Zufferey, R; Nagy, D; Mandel, R J; Naldini, L; and Trono, D. “Multiply attenuated lentiviral vector achieves efficient gene delivery in vivo. Nat. Biotechnol., 15: 871-875, 1997.
40. Aiken, C. “Pseudotyping human immunodeficiency virus type 1 (HIV-1) by the glycoprotein of vesicular stomatitis virus targets HIV-1 entry to an endocytic pathway and suppresses both the requirement for Nef and the sensitivity to cyclosporin A,” J. Virol., 71:5871-5877, 1997.
41. Xiao X, Li J, McCown T J, Samulski R J. “Gene transfer by adeno-associated virus vectors into the central nervous system,” Exp. Neurol., 144:113-124, 1997.
42. Rabinowitz, J E; and Samulski, R J. “Building a better vector: the manipulation of AAV virions,” Virology, 278:301-308, 2000.
43. Monahan, P E; and Samulski, RJ. “Adeno-associated virus vectors for gene therapy: more pros than cons?” Mol. Med. Today, 6:433-440, 2000.
44. Xiao, X; Li, J; and Samulski, RJ. “Production of high-titer recombinant adeno-associated virus vectors in the absence of helper adenovirus,” J. Virol., 72:2224-2232, 1998.
45. Ferrari, F K; Xiao, X; McCarty, D; and Samulski, R J. “New developments in the generation of Ad-free, high-titer rAAV gene therapy vectors,” Nat. Med., 3:1295-1297, 1997.
46. Collaco, R F; Cao, X; and Trempe J P. “A helper virus-free packaging system for recombinant adeno-associated virus vectors,” Gene, 238:397-405, 1999.
47. Abbas, A K; Lichtman, A H; and Pober, J S. Cellular and Molecular Immunology, pp. 79-100 (W. B. Saunders Co., Philadelphia, Pa., 2000).
48. Ghivizzani, S C; Lechman, E R; Tio, C; Mule, K M; Chada, S; McCormack, J E; Evans, C H; and Robbins, PD. “Direct retrovirus-mediated gene transfer to the synovium of the rabbit knee: implications for arthritis gene therapy,” Gene Ther., 4:977-982, 1997.
49. Ghivizzani, S C; Kang, R; Georgescu, H I; Lechman, E R; Jaffurs, D; Engle, J M; Watkins, S C; Tindal, M H; Suchanek, M K; McKenzie, L R; Evans, C H; and Robbins, P D. “Constitutive intra-articular expression of human IL-1 beta following gene transfer to rabbit synovium produces all major pathologies of human rheumatoid arthritis, J. Immunol., 159:3604-3612, 1997.
50. Gouze E, Pawliuk R, Pilapil C, Gouze J- N, Fleet C, Palmer G D, Evans C H, Leboulch P, Ghivizzani S C. “In vivo gene delivery to synovium by lentiviral vectors,” Mol, Ther., 5(4):397-404, 2002.
51. Baltzer, A W; Lattermann, C; Whalen, J D; Wooley, P; Weiss, K; Grimm, M; Ghivizzani, S C; Robbins, P D; and Evans, C H. “Genetic enhancement of fracture repair: healing of an experimental segmental defect by adenoviral transfer of the BMP-2 gene,” Gene Ther., 7(9):734-739, 2000.
52. Robbins, P D; Tahara, H; Mueller, G; Hung, G; Bahnson, A; Zitvogel, L; Galea-Lauri, J; Ohashi, T; Patrene, K; Boggs, S S; et al. “Retroviral vectors for use in human gene therapy for cancer, Gaucher disease, and arthritis,” Ann. NY Acad. Sci., 716:72-88; 1994.
53. Malim, M H; Hauber, J; Fenrick, R; and Cullen, B R. “Immunodeficiency virus rev trans-activator modulates the expression of the viral regulatory genes,” Nature, 335:181-183, 1988.
54. Yee, J K; Miyanohara, A; LaPorte, P; Bouic, K; Burns, J C; and Friedmann, T. “A general method for the generation of high-titer, pantropic retroviral vectors: highly efficient infection of primary hepatocytes,” Proc. Natl. Acad Sci. USA, 91:9564-9568, 1994.
55. Pawliuk, R; Kay, R; Lansdorp, P; and Humphries, R K. “Selection of retrovirally transduced hematopoietic cells using CD24 as a marker of gene transfer,” Blood, 84:2868-2877, 1994.
56. Grimm, D; Kern, A; Rittner, K; and Kleinschmidt, JA. “Novel tools for production and purification of recombinant adenoassociated virus vectors,” Hum. Gene Ther., 9:2745-2760, 1998.
57. Zolotukhin, S; Byme, B J; Mason, E; Zolotukhin, I; Potter, M; Chesnut, K; Summerford, C; Samulski, R J; and Muzyczka, N. “Recombinant adeno-associated virus purification using novel methods improves infectious titer and yield,” Gene Ther., 6:973-985, 1999.
58. Evans, CH; Gouze, J N; Gouze, E; Robbins, P D; and Ghivizzani, S C. “Osteoarthritis gene therapy,” Gene Ther., 11:379-389, 2004.
59. Evans, C H; Ghivizzani, S C; Herndon, J H; and Robbins, P D. “Gene therapy for the treatment of musculoskeletal diseases,” J. Am. Acad Orthop. Surg, 13:230-242, 2005.
60. McCarty, D M; Monahan, P E; and Samulski, R J. “Self-complementary recombinant adeno-associated virus (scAAV) vectors promote efficient transduction independently of DNA synthesis,” Gene Ther., 8:1248-1254, 2001.
61. McCarty, D M; Fu, H; Monahan, P E; Toulson, C E; Naik, P; and Samulski, RJ. “Adeno-associated virus terminal repeat (TR) mutant generates self-complementary vectors to overcome the rate-limiting step to transduction in vivo,” Gene Ther., 10:2112-2118, 2003.
62. Makarov S S, Olsen J C, Johnston W N, Anderle S K, Brown R R, Baldwin A S Jr, et al. “Suppression of experimental arthritis by gene transfer of interleukin 1 receptor antagonist cDNA,” Proc. Natl. Acad Sci. USA, 93:402-6, 1996.
63. Evans et al. “Gene therapy for arthritis: What next?” Arthritis Rheum., 54(6):1714-1729, 25 May 2006.
64. Gouze et al., “Transgene persistence and cell turnover in the diarthrodial joint: implications for gene therapy of chronic joint diseases,” Molec. Ther., 15(6),1114-1120, 2007.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for providing to a mammal in need thereof, a therapeutically-effective amount of a mammalian Interleukin-1 Receptor Antagonist peptide or polypeptide, said method comprising at least the step of:
(a) introducing into a plurality of cells or in a selected tissue of said mammal, a composition comprising a recombinant self-complimentary adeno-associated viral vector, wherein said vector comprises a nucleic acid segment that encodes a biologically-active mammalian IL-1Ra peptide or polypeptide, operably linked to a promoter that is capable of expressing said nucleic acid segment in a mammalian host cell that comprises said vector, in an amount, and for a time effective, to provide said mammal with said therapeutically-effective amount of said mammalian Interleukin-1 Receptor Antagonist peptide or polypeptide.

2. The method of claim 1, wherein said mammal has, is diagnosed with, or is at risk for developing arthritis, osteoarthritis, rheumatoid arthritis, a disease of the joint, or an articular or peri-articular injury, defect or dysfunction.

3. The method of claim 1, wherein said composition is administered to said mammal intramuscularly, intravenously, subcutaneously, intra-articularly, or peri-articularly.

4. The method of claim 3, wherein said composition is administered to the joint, synovium, subsynovium, joint capsule, tendon, ligament, cartilage or a peri-articular muscle of said mammal.

5. The method of claim 1, wherein said composition is directly injected into a first joint or first periarticular space of said mammal.

6. The method of claim 1, wherein said composition is formulated for administration to a human.

7. The method of claim 6, wherein said human is diagnosed with a joint injury, trauma, defect, disease, disorder, or dysfunction.

8. The method of claim 7, wherein said human is diagnosed with arthritis, osteoarthritis, neuromuscular disease, autoimmune disorder, or a joint injury or defect.

9. The method of claim 1, wherein said promoter is selected from the group consisting of a CMV promoter, a β-actin promoter, an EF1 promoter, a U1a promoter, a Tet-inducible promoter, a VP16-LexA promoter, a U1b promoter, and a joint-specific promoter.

10. The method of claim 9, wherein said β-actin promoter is a chicken M-actin promoter.

11. The method of claim 1, wherein said composition comprises a double-stranded adeno-associated viral particle or virion.

12. The method of claim 1, wherein said composition comprises a plurality of adeno-associated viral particles, wherein at least one of said particles comprises a recombinant self-complimentary adeno-associated viral vector that encodes said peptide or polypeptide.

13. The method of claim 1, wherein said composition further comprises at least a second therapeutic compound.

14. The method of claim 1, wherein said composition further comprises a liposome, lipid carrier, lipid complex, microsphere, microparticle, nanosphere, or nanoparticle.

15. The method of claim 1, wherein said composition is introduced into said cells or said tissue ex vivo; and further wherein said method comprises the additional step of introducing the resulting ex vivo cells or tissue that comprise said composition into at least a first tissue, joint, or peri-articular region of said mammal.

16. The method of claim 1, wherein said nucleic acid segment encodes a biologically-active mammalian IL-1Ra peptide or polypeptide comprising an amino acid sequence that is at least about 95% identical to the polypeptide sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO: 18, SEQ ID NO: 19, SEQ ID NO:20, or SEQ ID NO:21.

17. The method of claim 16, wherein said nucleic acid segment encodes a biologically-active mammalian IL-1Ra peptide or polypeptide comprising an amino acid sequence that is at least about 98% identical to the polypeptide sequence of any one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:19, SEQ ID NO:20, or SEQ ID NO:21.

18. The method of claim 17, wherein said nucleic acid segment encodes a biologically-active mammalian IL-1Ra peptide or polypeptide comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:19, SEQ ID NO:20, and SEQ ID NO:21.

19. The method of claim 16, wherein said nucleic acid segment encodes a biologically-active mammalian IL-1Ra peptide or polypeptide comprising an amino acid sequence that is at least about 95% identical to the polypeptide sequence of SEQ ID NO:2.

20. The method of claim 19, wherein said nucleic acid segment encodes a biologically-active mammalian IL-1Ra peptide or polypeptide comprising an amino acid sequence that is at least about 98% identical to the polypeptide sequence of SEQ ID NO:2.

21. The method of claim 20, wherein said nucleic acid segment encodes a biologically-active mammalian IL-1Ra peptide or polypeptide comprising the amino acid sequence of SEQ ID NO:2.

22. The method of claim 16, wherein said nucleic acid segment comprises a nucleotide sequence that is at least about 95% identical to the polynucleotide sequence of SEQ ID NO: 1.

23. The method of claim 22, wherein said nucleic acid segment comprises a nucleotide sequence that is at least about 98% identical to the polynucleotide sequence of SEQ ID NO: 1.

24. A method for providing directly to a mammalian joint or periarticular space, a therapeutically-effective amount of a biologically-active IL-1Ra peptide or polypeptide, said method comprising directly injecting into at least a first joint or periarticular space of said mammal an effective amount of a composition comprising:
a recombinant self-complimentary adeno-associated viral vector that comprises a nucleic acid segment that encodes a biologically-active mammalian IL-1Ra peptide or polypeptide, operably linked to a promoter that is capable of expressing said nucleic acid segment in a mammalian host cell that comprises said vector, in an amount, and for a time effective, to directly provide to said mammalian joint or periarticular space, said therapeutically-effective amount of said mammalian Interleukin-1 Receptor Antagonist peptide or polypeptide.

25. A method for treating or ameliorating the symptoms of an articular defect, injury, disease or dysfunction in a mammal, said method comprising administering to said mammal a composition comprising: recombinant self-complimentary adeno-associated viral vector that comprises a nucleic acid segment encoding a biologically-active mammalian IL-1Ra peptide or polypeptide, said segment operably linked to at least a first promoter that is capable of expressing said nucleic acid segment in a mammalian host cell that comprises said vector; in an amount and for a time sufficient to treat or ameliorate the symptoms of said articular defect, injury, disease or dysfunction in said mammal.
